(19) United States
(12) Patent Application Publication
(10) Pub. No.: US 2025/0171858 A1
(43) Pub. Date: May 29, 2025

(54) ENRICHMENT OF CLINICALLY-RELEVANT NUCLEIC ACIDS

(71) Applicant: Centre for Novostics, Shatin (HK)

(72) Inventors: Yuk-Ming Dennis Lo, Homantin (CN); Kwan Chee Chan, Jordan (CN); Peiyong Jiang, Pak Shek Kok (CN); Qing Zhou, Tai Po (CN); Wenlei Peng, Shatin (CN)

(21) Appl. No.: 18/964,174

(22) Filed: Nov. 29, 2024

Related U.S. Application Data
(60) Provisional application No. 63/604,167, filed on Nov. 29, 2023.

(51) Int. Cl. C12Q 1/6886; C12Q 1/6806; C12Q 1/686; C12Q 1/6874; G16B 20/20; G16B 30/10; G16H 50/20

(52) U.S. Cl. CPC C12Q 1/6886; C12Q 1/6806; C12Q 1/686; C12Q 1/6874; G16B 20/20; G16B 30/10; G16H 50/20; C12Q 2600/154

(57) ABSTRACT

Techniques are provided for enriching samples for clinically-relevant cell-free (cfDNA), e.g., for downstream analysis. For example, cell-free DNA molecules having NCG end motifs can be selected, as such a subset has an increased proportion of the clinically-relevant cfDNA. Such NCG cfDNA molecules can be further selected based on size and/or location at tissue-specific hypomethylated sites for further enrichment. As another example, cell-free DNA molecules having CGN end motifs can be selected, as such a subset can have an increased proportion of the clinically-relevant cfDNA when located at tissue-specific hypermethylated sites. Such CGN cfDNA molecules can be further selected based on size.

### CROSS-REFERENCES TO RELATED APPLICATIONS

The present application claims priority from and is a non-provisional application of U.S. Provisional Application No. 63/604,167, entitled “Enrichment Of Clinically-Relevant Nucleic Acids” filed Nov. 29, 2023, the entire contents of which are herein incorporated by reference for all purposes.

### BACKGROUND

The detection of circulating cell-free fetal DNA (cfDNA) has been increasingly adopted in noninvasive prenatal testing (NIPT) since its discovery. Useful information and implications can be determined from the cfDNA for the identification and classification of conditions (e.g., diseases) in the fetal and other contexts (e.g., for cancer). However, samples (e.g., plasma or urine) often have a relatively low amount of clinically-relevant DNA (e.g. fetal or tumor DNA), making detection of a condition difficult. Accordingly, it would be beneficial to increase the fraction of the clinically-relevant cfDNA in a sample, thereby making analysis more accurate, enabling smaller samples, and/or require less effort, e.g., fewer reagents required.

### BRIEF SUMMARY

Embodiments of the present disclosure provide for enriching samples for clinically-relevant cell-free (cfDNA), e.g., for downstream analysis. For example, cell-free DNA molecules having NCG end motifs can be selected, as such a subset has an increased proportion of the clinically-relevant cfDNA. Such NCG cfDNA molecules can be further selected based on size and/or location at tissue-specific hypomethylated sites for further enrichment. As another example, cell-free DNA molecules having CGN end motifs can be selected, as such a subset can have an increased proportion of the clinically-relevant cfDNA when located at tissue-specific hypermethylated sites. Such CGN cfDNA molecules can be further selected based on size.

One general aspect includes a method of enriching a biological sample of a subject for clinically-relevant DNA. The method also includes analyzing a plurality of cell-free DNA molecules from the biological sample of the subject. Analyzing each cell-free DNA molecule of the plurality of cell-free DNA molecules can include: determining an end sequence motif of at least one end of the cell-free DNA molecule. An end of the cell-free DNA molecule can have a first position at an outermost position, a second position that is next to the first position, and a third position that is next to the second position. The method also includes identifying a first group of the plurality of cell-free DNA molecules having a set of one or more end sequence motifs. The set of one or more end sequence motifs can have C at the second position and G at the third position. The first group of the plurality of cell-free DNA molecules can be used to enrich the biological sample for the clinically-relevant DNA.

Another general aspect includes a method of analyzing a biological sample of a subject for genomic deletions or amplifications. The method also includes analyzing a plurality of cell-free DNA molecules from the biological sample of the subject. Analyzing a cell-free DNA molecule can include: determining an end sequence motif of at least one end of the cell-free DNA molecule. An end of the cell-free DNA molecule can have a first position at an outermost position, a second position that is next to the first position, and a third position that is next to the second position. The method also includes identifying a first group of the plurality of cell-free DNA molecules having a set of one or more end sequence motifs. The set of one or more end sequence motifs can have C at the second position and G at the third position. The method also includes determining locations of the first group of the plurality of cell-free DNA molecules in a reference genome. The method also includes identifying, using the locations, a first subgroup of the first group of cell-free DNA molecules that are located in a chromosomal region including one or more specified loci. The method also includes calculating a first value of the first subgroup of cell-free DNA molecules. The first value can define a characteristic of the first subgroup of cell-free DNA molecules. The method also includes comparing the first value to a reference value to determine a classification of whether the chromosomal region exhibits a deletion or an amplification in the clinically-relevant DNA.

Another general aspect includes a method of enriching a biological sample of a subject for clinically-relevant DNA. The method also includes analyzing a plurality of cell-free DNA molecules from the biological sample of the subject. Analyzing a cell-free DNA molecule can include: determining a location of the cell-free DNA molecule in a reference genome; determining an end sequence motif of at least one end of the cell-free DNA molecule. An end of the cell-free DNA molecule has a first position at an outermost position, a second position that is next to the first position, and a third position that is next to the second position. The method also includes identifying a first group of the plurality of cell-free DNA molecules that (1) have a set of one or more end sequence motifs, where the set of one or more end sequence motifs have C at the first position and G at the second position, and (2) are located at a set of sites that are hypermethylated in the clinically-relevant DNA. The method also includes using the first group of the plurality of cell-free DNA molecules to enrich the biological sample for the clinically-relevant DNA.

Another general aspect includes a method of analyzing a biological sample of a subject for genomic deletions or amplifications. The method also includes analyzing a plurality of cell-free DNA molecules from the biological sample of the subject. Analyzing a cell-free DNA molecule can include: determining a location of the cell-free DNA molecule in a reference genome; and determining an end sequence motif of at least one end of the cell-free DNA molecule. An end of the cell-free DNA molecule has a first position at an outermost position, a second position that is next to the first position, and a third position that is next to the second position. The method also includes identifying a first group of the plurality of cell-free DNA molecules that (1) have a set of one or more end sequence motifs, where the set of one or more end sequence motifs have C at the first position and G at the second position, and (2) are located at a set of sites that are hypermethylated in the clinically-relevant DNA, where a chromosomal region includes the set of sites. The method also includes calculating a first value of the first group of cell-free DNA molecules, the first value defining a characteristic of the first group of cell-free DNA molecules. The method also includes comparing the first value to a reference value to determine a classification of whether the chromosomal region exhibits a deletion or an amplification in the clinically-relevant DNA.

These and other embodiments of the disclosure are described in detail below. For example, other embodiments are directed to systems, devices, and computer readable media associated with methods described herein.

Reference to the remaining portions of the specification, including the drawings and claims, will realize other features and advantages of the present disclosure. Further features and advantages of the present disclosure, as well as the structure and operation of various embodiments of the present disclosure, are described in detail below with respect to the accompanying drawings. In the drawings, like reference numbers can indicate identical or functionally similar elements.

### Terms

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells. “Reference tissues” can correspond to tissues used to determine tissue-specific methylation levels or tissue-specific fragmentation patterns. Multiple samples of a same tissue type from different individuals may be used to determine a tissue-specific methylation level for that tissue type.

A “biological sample” refers to any sample that is taken from a subject (e.g., a human or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia) and contains one or more nucleic acid molecule(s) of interest (e.g., DNA and/or RNA). The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, peritoneal dialysate, discharge fluid from the nipple, aspiration fluid from different parts of the body (e.g., thyroid, breast), intraocular fluids (e.g., the aqueous humor), amniotic fluid, etc. Stool samples can also be used. In various embodiments, the majority of DNA in a biological sample (e.g., that has been enriched for cell-free DNA, such as a plasma sample obtained via a centrifugation protocol) can be cell-free, e.g., greater than 50%, 60%, 70%, 80%, 90%, 95%, or 99% of the DNA can be cell-free. A centrifugation protocol for enriching cell-free DNA from a biological sample can include, for example, centrifuging the biological sample at 1,600 g×10 minutes, obtaining the fluid part of the centrifuged sample, and re-centrifuging at for example, 16,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed. Any amount described herein can be any of the numbers listed above. Examples sizes of a sample can include 30, 50, 100, 200, 300, 500, 1,000, 5,000, or 10,000 or more nanograms, or 1, 2, 3, 4, 5, 6, 7, 8, 9, or 10 ml.

“Clinically-relevant DNA” can refer to DNA of a particular tissue source that is to be measured, e.g., to determine a fractional concentration of such DNA or to classify a phenotype of a sample (e.g., plasma). Examples of clinically-relevant DNA are fetal DNA in maternal plasma or tumor DNA in a patient's plasma or other sample with cell-free DNA. Another example includes the measurement of the amount of graft-associated DNA in the plasma, serum, or urine of a transplant patient. A further example includes the measurement of the fractional concentrations of hematopoietic and nonhematopoietic DNA in the plasma of a subject, or fractional concentration of a liver DNA fragments (or other tissue) in a sample or fractional concentration of brain DNA fragments in cerebrospinal fluid.

The term “fractional fetal DNA concentration” is used interchangeably with the terms “fetal DNA proportion” and “fetal DNA fraction,” and refers to the proportion of fetal DNA molecules that are present in a biological sample (e.g., maternal plasma or serum sample) that is derived from the fetus (Lo et al, Am J Hum Genet. 1998; 62:768-775; Lun et al, Clin Chem. 2008; 54:1664-1672). Similarly, tumor fraction or tumor DNA fraction can refer to the fractional concentration of tumor DNA in a biological sample, or tissue fraction can refer to the fractional concentration of DNA from one or more particular tissue(s), e.g., from a transplant organ.

The term “fragment” (e.g., a DNA or an RNA fragment), as used herein, can refer to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins. A nucleic acid fragment can be a linear fragment or a circular fragment. A tumor-derived nucleic acid can refer to any nucleic acid released from a tumor cell, including pathogen nucleic acids from pathogens in a tumor cell. As part of an analysis of a biological sample, a statistically significant number of fragments can be analyzed, e.g., at least 1,000 fragments can be analyzed. As other examples, at least 5,000, 10,000 or 50,000 or 100,000 or 500,000 or 1,000,000 or 5,000,000 fragments, or more, can be analyzed, and such fragments can be randomly selected or selected according to one or more criteria. A same number of reactions can be analyzed, e.g., for digital PCR or sequencing.

The term “assay” generally refers to a technique for determining a property of a nucleic acid or a sample of nucleic acids (e.g., a statistically significant number of nucleic acids), as well as a property of the subject from which the sample was obtained. An assay (e.g., a first assay or a second assay) generally refers to a technique for determining the quantity of nucleic acids in a sample, genomic identity of nucleic acids in a sample, the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art may be used to detect any of the properties of nucleic acids mentioned herein. Properties of nucleic acids include a sequence, quantity, genomic identity, copy number, a methylation state at one or more nucleotide positions, a size of the nucleic acid, a mutation in the nucleic acid at one or more nucleotide positions, and the pattern of fragmentation of a nucleic acid (e.g., the nucleotide position(s) at which a nucleic acid fragments). The term “assay” may be used interchangeably with the term “method”. An assay or method can have a particular sensitivity and/or specificity (e.g., based on selection of one or more cutoff values), and their relative usefulness as a diagnostic tool can be measured using Receiver Operating Characteristic (ROC) Area-Under-the-Curve (AUC) statistics.

A “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example probe-based techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000, 50,000, 100,000, 500,000, 1,000,000, or 5,000,000 sequence reads, or more, can be analyzed. Additionally, amounts of sequence reads determined for embodiments of the present disclosure can be at least 1,000, 5,000, 10,000, 50,000, 100,000, 500,000, 1,000,000, or 5,000,000.

The term “mapping” or “aligning” refers to a process that relates a sequence to a location or coordinate (e.g., a genomic coordinate) in a reference (e.g., a reference genome) having a known reference sequence, where the sequence is similar to the known reference sequence at the location in the reference. The degree of similarity can be measured or reported in terms of a “mapping quality.” In one example of a mapping quality used herein, a mapping quality of X for a sequence with respect to a reported location or coordinate in a reference indicates that the probability of the sequence mapping to a different location is no greater than 10\hat{ }(−X/10). For instance, a mapping quality of 30 indicates a less than 0.1% probability of the sequence mapping to an alternate location.

A “reference genome” or “reference sequence” may be an entire genome sequence of a reference organism, one or more portions of a reference genome that may or may not be contiguous, a consensus sequence of many reference organisms, a compilation sequence based on different components of different organisms, or any other appropriate reference sequence. As examples, a reference genome/sequence can be at least 1,000, 10,000, 50,000, 100,000, 500,000, 1,000,000, 5,000,000, 10,000,000, 50,000,000, 100,000,000, 500,000,000, one billions, or 3 billion nucleotides long, e.g., a full human genome or a repeat masked human genome. A reference may also include information regarding variations of the reference known to be found in a population of organisms.

A “site” (also called a “genomic site”) corresponds to a single site, which may be a single base position or a group of correlated base positions, e.g., a CpG site, TSS site, DNase hypersensitivity site, or larger group of correlated base positions. A “locus” may correspond to a region that includes multiple sites. A locus can include just one site, which would make the locus equivalent to a site in that context. Various number of regions, sites, or loci can be analyzed, e.g., 50, 100, 200, 500, 1,000, 5,000, 10,000, 50,000, 100,000, 500,000, one million, or more. Various techniques can determine a DNA molecule is located at one or more genomic positions in a reference genome, e.g., alignment of a sequence read to the reference genome or using position-specific probes. The position determination can be to some or all of the reference genome, e.g., if only part of the genome is being analyzed. As examples, the amount of the genome analyzed can be greater than 0.01%, 0.1%, 1%, 5%, 10%, or 50%. A “cutting site” can refer to a location that DNA was cut by a nuclease, thereby resulting in a DNA fragment.

A sequence read can include an “ending sequence” associated with an end of a fragment. The ending sequence can correspond to the outermost N bases of the fragment, e.g., 1-30 bases at the end of the fragment. If a sequence read corresponds to an entire fragment, then the sequence read can include two ending sequences. When paired-end sequencing provides two sequence reads that correspond to the ends of the fragments, each sequence read can include one ending sequence.

A “sequence motif” may refer to a short, recurring pattern of bases in DNA fragments (e.g., cell-free DNA fragments). A sequence motif can occur at an end of a fragment, and thus be part of or include an ending sequence. An “end motif” (also referred to as a “end sequence motif”) can refer to a sequence motif for an ending sequence that preferentially occurs at ends of DNA fragments, potentially for a particular type of tissue. An end motif may also occur just before or just after ends of a fragment, thereby still corresponding to an ending sequence. A nuclease can have a specific cutting preference for a particular end motif, as well as a second most preferred cutting preference for a second end motif. The number of nucleotides (nt) at the fragment ends used for analysis could be, for example, but not limited to, 1 nt, 2 nt, 3 nt, 4 nt, 5 nt, 6 nt, 7 nt, 8 nt, 9 nt, and 10 nt or above. In some embodiments, the fragment end motif could be defined by one or more nucleotides across positions nearby the end of a fragment. The fragment end motif could be defined by one or more nucleotides in a reference genome surrounding the genomic locus to which the end of a fragment is aligned. Various numbers of motifs can be used, e.g., at least 1, 2, 3, 4, 5, 6, 7, 8, 9, 10, 20, 30, 40, 50 60, 70, 80, 90, 100, 150, 200, 250, or 256 end motifs.

A “sequence motif pair” or “end motif pair” may refer to a pair of end motifs of a particular DNA fragment. For example, a DNA fragment having an A at the 5′ end of one strand and an A at the 5′ end of the other strand can be defined as having a sequence motif pair of A< >A. Other lengths of sequence motifs can be used. Different paired combinations of end motifs can be referred to as different types of fragments. End motif pairs may include end motifs that are the same length, e.g., both 1-mers or both 2-mers, but may also include end motifs that are of different lengths, e.g., one end is a 2-mer and the other end is composed of 1-mers. End motif pairs may also include one or more bases past the end of the DNA fragment, e.g., as determined by aligning to a reference genome. Such an instance can use the nomenclature t|A, where T occurs just before a cutting site at the 5′ end, and A occurs after the cutting site.

A “relative frequency” (also referred to just as “frequency”) may refer to a proportion (e.g., a percentage, fraction, or concentration). In particular, a relative frequency of a particular end motif (e.g., A, CG, TAG, etc.) or end motif pair (e.g., A< >A) can provide a proportion of cell-free DNA fragments that have that end motif or that particular pair end motif pair.

The term “alleles” refers to alternative DNA sequences at the same physical genomic locus, which may or may not result in different phenotypic traits. In any particular diploid organism, with two copies of each chromosome (except the sex chromosomes in a male human subject), the genotype for each gene comprises the pair of alleles present at that locus, which are the same in homozygotes and different in heterozygotes. A population or species of organisms typically include multiple alleles at each locus among various individuals. A genomic locus where more than one allele is found in the population is termed a polymorphic site. Allelic variation at a locus is measurable as the number of alleles (i.e., the degree of polymorphism) present, or the proportion of heterozygotes (i.e., the heterozygosity rate) in the population. As used herein, the term “polymorphism” refers to any inter-individual variation in the human genome, regardless of its frequency. Examples of such variations include, but are not limited to, single nucleotide polymorphism, simple tandem repeat polymorphisms, insertion-deletion polymorphisms, mutations (which may be disease causing) and copy number variations. The term “haplotype” as used herein refers to a combination of alleles at multiple loci that are transmitted together on the same chromosome or chromosomal region. A haplotype may refer to as few as one pair of loci or to a chromosomal region, or to an entire chromosome or chromosome arm.

The terms “size profile” and “size distribution” generally relate to the sizes of DNA fragments in a biological sample. A size profile may be a histogram that provides a distribution of an amount of DNA fragments at a variety of sizes. Various statistical values (also referred to as size parameters or just parameter) can distinguish one size profile to another. One parameter (e.g., a statistical value) is the percentage of DNA fragment of a particular size or range of sizes relative to all DNA fragments or relative to DNA fragments of another size or range.

“DNA methylation” in mammalian genomes typically refers to the addition of a methyl group to the 5′ carbon of cytosine residues (i.e., 5-methylcytosines or 5mC) among CpG dinucleotides. DNA methylation may occur in cytosines in other contexts, for example CHG and CHH, where H is adenine, cytosine or thymine. Cytosine methylation may also be in the form of 5-hydroxymethylcytosine. Non-cytosine methylation, such as N6-methyladenine, has also been reported. Other types of methylation have been found on cytosines, adenines, thymines and guanines, such as 4mC (N4-methylcytosine), 5hmC (5-hydroxymethylcytosine), 5fC (5-formylcytosine), 5caC (5-carboxylcytosine), 1 mA (N1-methyladenine), 3 mA (N3-methyladenine), 7 mA (N7-methyladenine), 3mC (N3-methylcytosine), 2mG (N2-methylguanine), 6mG (06-methylguanine), 7mG (N7-methylguanine), 3mT (N3-methylthymine), and 4mT (04-methylthymine). In vertebrate genomes, 5mC is the most common type of base methylation, followed by that for guanine (i.e., in the CpG context).

The “methylation index” for each genomic site (e.g., a CpG site) can refer to the proportion of DNA fragments (e.g., as determined from sequence reads or probes) showing methylation at the site over the total number of reads covering that site. A “methylation status” can refer to whether a particular site is methylated at a particular site of a DNA fragment or whether a particular site in a genome has a particular differential methylation status, e.g., hypermethylation or hypomethylation. A “read” can include information (e.g., methylation status at a site) obtained from a DNA fragment. A read can be obtained using reagents (e.g., primers or probes) that preferentially hybridize to DNA fragments of a particular methylation status. Typically, such reagents are applied after treatment with a process that differentially modifies or differentially recognizes DNA molecules depending on their methylation status, e.g., bisulfite conversion, or methylation-sensitive restriction enzyme, or methylation binding proteins, or anti-methylcytosine antibodies, or single molecule sequencing techniques that recognize methylcytosines and hydroxymethylcytosines.

The “methylation density” of a region or a set of sites can refer to the number of reads at site(s) within the region (also referred to as a bin) or the set of sites showing methylation divided by the total number of reads covering the site(s) in the region or the set of sites. A region can include one or more sites of interest, including at least 1, 2, 3, 4, 5, 10, 20, 50, 100, 200, 500, and 1,000 sites. The site(s) may have specific characteristics, e.g., being CpG sites. Thus, the “CpG methylation density” of a region can refer to the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of cytosines not converted after bisulfite treatment (which corresponds to methylated cytosine) at CpG sites as a proportion of all CpG sites covered by sequence reads mapped to the 100-kb region. This analysis can also be performed for other bin sizes, e.g., 500 bp, 5 kb, 10 kb, 50-kb or 1-Mb, etc. A region could be the entire genome or a chromosome or part of a chromosome (e.g., a chromosomal arm). The methylation index of a CpG site is the same as the methylation density for a region when the region only includes that CpG site. The “proportion of methylated cytosines” can refer to the number of cytosine sites, “C's”, that are shown to be methylated (for example unconverted after bisulfite conversion) over the total number of analyzed cytosine residues, i.e., including cytosines outside of the CpG context, in the region. The methylation index, methylation density and proportion of methylated cytosines are examples of “methylation levels.” Apart from bisulfite conversion, other processes known to those skilled in the art can be used to interrogate the methylation status of DNA molecules, including, but not limited to enzymes sensitive to the methylation status (e.g. methylation-sensitive restriction enzymes), methylation binding proteins, single molecule sequencing using a platform sensitive to the methylation status (e.g. nanopore sequencing (Schreiber et al. Proc Natl Acad Sci USA 2013; 110: 18910-18915) and by the Pacific Biosciences single molecule real time analysis (Tse et al. Proc Natl Acad Sci USA 2021; 118: e2019768118).

A “methylation level” is an example of a relative abundance, e.g., between methylated DNA molecules (e.g., at one or more particular sites) and other DNA molecules (e.g., all other DNA molecules or just unmethylated DNA molecules at the one or more particular sites). The amount of other DNA molecules can act as a normalization factor. As another example, an intensity of methylated DNA molecules (e.g., fluorescent or electrical intensity) relative to intensity of all or unmethylated DNA molecules at one or more sites can be determined. The relative abundance can also include an intensity per volume. A methylation level can be determined using a methylation-aware assay such as methylation-aware sequencing or PCR. Example methylation-aware sequencing can include bisulfite sequencing or single molecule techniques, e.g., using nanopores.

A differentially methylated region (DMR) is a genomic region (e.g., set of sites) with different DNA methylation level across two or more biological samples. The different DNA methylation level may be defined by the certain difference in methylation index or density, such as but not limited to 1%, 5%, 10%, 20%, 30%, 40%, 50%, 60%, 70%, 80%, 90%, 95%, 99%, etc. A differentially methylated site (DMS) may be defined in a similar manner.

The term “hypomethylation” can refer to a site or set of sites (e.g., a region) that has below a specified threshold for a methylation level, e.g., at or below 50%, 45%, 40%, 35%, 30%, 25%, or 20% for the methylation level. A site in a genome may be considered unmethylated if the methylation level is below a threshold. The term “hypermethylation” can refer to a site or set of sites (e.g., a region) that has above a specified value for a methylation level, e.g., at or above 95%, 90%, 80%, 75%, 70%, 65%, or 60% for the methylation level. A site in a genome may be considered methylated if the methylation level is greater than a threshold. Hypomethylation or hypermethylation can occur for a particular tissue or across a set of tissues.

The term “sequencing depth” refers to the number of times a locus is covered by a sequence read aligned to the locus. The locus could be as small as a nucleotide, or as large as a chromosome arm, or as large as the entire genome. Sequencing depth can be expressed as 50×, 100×, etc., where “×” refers to the number of times a locus is covered with a sequence read. Sequencing depth can also be applied to multiple loci, or the whole genome, in which case × can refer to the mean number of times the loci or the haploid genome, or the whole genome, respectively, is sequenced. Ultra-deep sequencing can refer to at least 100× in sequencing depth.

The term “parameter” as used herein means a numerical value that characterizes a quantitative data set and/or a numerical relationship between quantitative data sets. For example, a difference or a ratio (or function of a ratio) between a first amount of a first nucleic acid sequence and a second amount of a second nucleic acid sequence is a parameter. As examples, a direct ratio of x/y is a separation value, as well as x/(x+y). The separation value can include other factors, e.g., multiplicative factors. As other examples, a difference or ratio of functions of the values can be used, e.g., a difference or ratio of the natural logarithms (ln) of the two values. A separation value can include a difference and a ratio. The parameter can be compared to a reference value (e.g., a threshold or cutoff) to determine any classification described herein, e.g., with respect to fetal, cancer, or transplant analysis. A normalized amount, e.g., a relative frequency, is an example of a parameter.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1), including probabilities. Different techniques for determining a classification can be combined to obtain a final classification from the initial or intermediate classification for each of the different techniques, e.g., by majority vote or a requirement that all initial/intermediate classifications are the same (e.g., positive).

The term “sequence imbalance” or “aberration” as used herein means any significant deviation as defined by at least one cutoff value in a quantity of the clinically relevant chromosomal region from a reference quantity. A sequence imbalance can include chromosome dosage imbalance, allelic imbalance, mutation dosage imbalance, copy number imbalance, haplotype dosage imbalance, and other similar imbalances. As an example, an allelic imbalance can occur when a tumor has one allele of a gene deleted or one allele of a gene amplified or differential amplification of the two alleles in its genome, thereby creating an imbalance at a particular locus in the sample. As another example, a patient could have an inherited mutation in a tumor suppressor gene. The patient could then go on to develop a tumor in which the non-mutated allele of the tumor suppressor gene is deleted. Thus, within the tumor, there is mutation dosage imbalance. When the tumor releases its DNA into the plasma of the patient, the tumor DNA will be mixed in with the constitutional DNA (from normal cells) of the patient in the plasma. An aberration can include a deletion or amplification of a chromosomal region.

The terms “cutoff” and “threshold” refer to predetermined numbers used in an operation. For example, a cutoff size can refer to a size above which fragments are excluded. As another example, a threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g., recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g., symptoms or other positive tests), has cancer. A level for various types of cancer can be determined, e.g., carcinoma or sarcoma, melanoma, lymphoma, and leukemia, as well as in various tissue of origin, including by way of example: breast, lung, liver, colon, pancreas, stomach, bone, blood, head and neck (e.g., head and neck squamous cell carcinoma), throat, bladder, kidney, prostate, uterine, rectal, bile duct, brain, eye, esophageal, ovarian, oral cavity, Nasopharyngeal, thyroid, urethral, testicular, vaginal, and pituitary.

A “level of pathology” can refer to the amount, degree, or severity of pathology associated with an organism, where the level can be as described above for cancer. Another example of pathology is a rejection of a transplanted organ. Other example pathologies can include autoimmune attack (e.g., lupus nephritis damaging the kidney or multiple sclerosis damaging the central nervous system), inflammatory diseases (e.g., hepatitis), fibrotic processes (e.g., cirrhosis), fatty infiltration (e.g., fatty liver diseases), degenerative processes (e.g., Alzheimer's disease) and ischemic tissue damage (e.g., myocardial infarction or stroke). A heathy state of a subject can be considered a classification of no pathology.

The terms “control”, “control sample”, “background sample,” “reference”, “reference sample”, “normal”, and “normal sample” may be interchangeably used to generally describe a sample that does not have a particular condition or is otherwise healthy. In an example, a no-template control (NTC) sample with contaminant DNA can be considered as a reference sample. In another example, the reference sample is a sample taken from a subject without an infection. A reference sample may be obtained from the subject, or from a database. The reference generally refers to a reference genome that is used to map sequence reads obtained from sequencing a sample from the subject. A reference genome generally refers to a haploid or diploid genome to which sequence reads from the biological sample can be aligned and compared. For a haploid genome, there is only one nucleotide at each locus. For a diploid genome, heterozygous loci can be identified, with such a locus having two alleles, where either allele can allow a match for alignment to the locus. A reference genome can be a reference microbe genome that corresponds to a particular microbe species, e.g., by including one or more microbe genomes.

A “machine learning model” (ML model) can refer to a software module configured to be run on one or more processors to provide a classification or numerical value of a property of one or more samples. An ML model can include various parameters (e.g., for coefficients, weights, thresholds, functional properties of function, such as activation functions). As examples, an ML model can include at least 10, 100, 1,000, 5,000, 10,000, 50,000, 100,000, or one million parameters. An ML model can be generated using sample data (e.g., training samples) to make predictions on test data. Various number of training samples can be used, e.g., at least 10, 100, 1,000, 5,000, 10,000, 50,000, 100,000, or at least 200,000 training samples. One example is an unsupervised learning model such as hidden Markov model (HMM), clustering (e.g., hierarchical clustering, k-means, mixture models, model-based clustering, density-based spatial clustering of applications with noise (DBSCAN), and OPTICS algorithm), approaches for learning latent variable models such as Expectation-maximization algorithm (EM), method of moments, and blind signal separation techniques (e.g., principal component analysis, independent component analysis, non-negative matrix factorization, singular value decomposition), and anomaly detection (e.g., local outlier factor and isolation forest). Another example type of model is supervised learning that can be used with embodiments of the present disclosure. Example supervised learning models may include different approaches and algorithms including analytical learning, statistical models, artificial neural network (e.g. including convolutional and/or transformer layers) that may have 1-10 layers as examples, recurrent neural network (e.g., long short term memory, LSTM), boosting (meta-algorithm), bootstrap aggregating (bagging) such as random forests, support vector machine (SVM), support vector (SVR), Bayesian statistics, case-based reasoning, decision tree learning, inductive logic programming, linear regression, logistic regression, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), ordinal classification, data pre-processing, handling imbalanced datasets, statistical relational learning, or Proaftn (a multicriteria classification algorithm), or an ensemble of any of these types. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range (e.g., range can be greater than or less than specified number), and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### BRIEF DESCRIPTION OF THE DRAWINGS

FIG. 1 shows examples for end motifs according to embodiments of the present disclosure.

FIG. 2 illustrates cutting positions relative to CpG sites (also referred to as CG sites) according to embodiments of the present disclosure.

FIG. 3 shows different types of cfDNA fragments, including NCG fragments and CGN fragments, which can be used to enrich a sample for clinically-relevant DNA.

FIGS. 4A-4B show the fetal fractional concentration of NCG-ended fetal DNA fragments, CGN-ended fetal DNA fragments, and all fragments fetal DNA.

FIGS. 5A-5B show the tumor fractional concentration of NCG-ended DNA fragments, CGN-ended DNA fragments, and all DNA fragments.

FIG. 6 shows different types of cfDNA fragments, including NCG fragments and CGN fragments, which were then analyzed at tissue-specific methylated CpG sites to enrich a sample for clinically-relevant DNA.

FIG. 7 shows boxplots illustrating the fractional concentration of NCG DNA fragments (placenta hypomethylated CpGs), NCG DNA fragments (placenta hypermethylated CpGs), CGN DNA fragments (placenta hypomethylated CpGs), CGN DNA fragments (placenta hypermethylated CpGs), and all DNA fragments (placenta hypomethylated CpGs).

FIGS. 8A-8B show tables of the fractional concentration of NCG DNA fragments (placenta hypomethylated CpGs), NCG DNA fragments (placenta hypermethylated CpGs), CGN DNA fragments (placenta hypomethylated CpGs), CGN DNA fragments (placenta hypermethylated CpGs), and all DNA fragments.

FIG. 9 shows the fractional concentration of NCG DNA fragments (HCC hypomethylated CpGs), NCG DNA fragments (HCC hypermethylated CpGs), CGN DNA fragments (HCC hypomethylated CpGs), CGN DNA fragments (HCC hypermethylated CpGs), and all DNA fragments.

FIG. 10 shows the fractional concentration of NCG tumor fragments (HCC hypomethylated CpGs), NCG DNA fragments (HCC hypermethylated CpGs), CGN DNA fragments (HCC hypomethylated CpGs), CGN DNA fragments (HCC hypermethylated CpGs), and all DNA fragments tumor.

FIG. 11 shows a series of selection steps, including NCG fragments separately having ACG, CCG, GCG, and TCG fragments, which were then analyzed at tissue-specific methylated CpG sites to enrich a sample for clinically-relevant DNA.

FIG. 12 shows boxplots of the fractional concentration of ACG DNA fragments, CCG DNA fragments, GCG DNA fragments, TCG DNA fragments, and all DNA fragments at placenta hypomethylated CpGs

FIGS. 13A-13B show tables of the fractional concentration of ACG DNA fragments, CCG DNA fragments, GCG DNA fragments, TCG DNA fragments, and all DNA fragments at placenta hypomethylated CpGs.

FIG. 14 illustrates the expression level of DNASE1L3 in placenta tissues and blood cells.

FIGS. 15A-15B show the fractional concentration of ACG DNA fragments, CCG DNA fragments, GCG DNA fragments, TCG DNA fragments, and all DNA fragments at HCC hypomethylated CpGs.

FIG. 16A illustrates the expression level of DNASE1L3 in HCC tumoral tissues and adjacent nontumoral liver tissues.

FIG. 16B illustrates the expression level of DNASE1 in HCC tumoral tissues and adjacent nontumoral liver tissues.

FIG. 17 shows a selection of NCG fragments and a size selection to enrich a sample for clinically-relevant DNA.

FIG. 18 shows boxplots of the fetal fraction for all DNA fragments, DNA fragments less than 150 bp, and NCG DNA fragments less than 150 bp.

FIGS. 19A-19B show tables of the fetal fraction for all DNA fragments, DNA fragments less than 150 bp, and NCG fragments less than 150 bp and relative change to all DNA fragments.

FIG. 20A shows the fetal fraction for all DNA fragments, DNA fragments less than 150 bp, and NCG DNA fragments less than 150 bp. FIG. 20B shows a table of the fetal fraction for all DNA fragments, DNA fragments less than 150 bp, and NCG fragments less than 150 bp and relative change to all DNA fragments.

FIG. 21 shows an example workflow for the sequencing library preparation by selectively amplifying cfDNA containing CCG end motifs in which the CG represents a tissue-specific hypomethylated CpG site.

FIGS. 22A-22B show simulated amount of reads for a Trisomy 21 diagnosis. The fetal fraction is fixed to be 5%, and the expected specificity is 100%.

FIG. 23 shows an example workflow using digital PCR.

FIG. 24 is a flowchart illustrating a method of enriching a biological sample of a subject for clinically-relevant DNA using one or more NCG end motifs.

FIG. 25 is a flowchart illustrating a method of analyzing a biological sample of a subject for genomic deletions or amplifications.

FIG. 26 is a flowchart illustrating a method of enriching a biological sample of a subject for clinically-relevant DNA using one or more CGN end motifs and tissue-specific hypermethylated sites.

FIG. 27 is a flowchart illustrating a method of analyzing a biological sample of a subject for genomic deletions or amplifications using one or more CGN end motifs and tissue-specific hypermethylated sites.

FIGS. 28A-28C show performance of CCG target sequencing in NIPT.

FIG. 29 shows a simulated ROC curves for detecting cancer copy number variation using whole genome sequencing, TCG target sequencing with size <150 bp, and TCG target sequencing with further region selection (i.e., cancer hypomethylated region) with 1 million reads.

FIG. 30 illustrates a measurement system according to an embodiment of the present disclosure.

FIG. 31 shows a block diagram of an example computer system usable with systems and methods according to embodiments of the present disclosure.

FIG. 32 illustrates the expression level of DNASE1L3 in tumoral tissues and adjacent nontumoral tissues in various of cancers. BLCA: Bladder Urothelial Carcinoma; BRCA: Breast invasive carcinoma; ESCA: Esophageal carcinoma; HNSC: Head and Neck squamous cell carcinoma; KIPAN: Kidney PAN cancer; KIRC: Kidney renal clear cell carcinoma; LIHC: Liver hepatocellular carcinoma; LUAD: Lung adenocarcinoma; LUSC: Lung squamous cell carcinoma; STAD: Stomach adenocarcinoma; STES: Stomach and Esophageal carcinoma; THCA: Thyroid carcinoma; UCEC: Uterine Corpus Endometrial Carcinoma.

FIG. 33 illustrates the expression level of DNASE1 in tumoral tissues and adjacent nontumoral tissues in various of cancers. BLCA: Bladder Urothelial Carcinoma; BRCA: Breast invasive carcinoma; ESCA: Esophageal carcinoma; HNSC: Head and Neck squamous cell carcinoma; KIPAN: Kidney PAN cancer; KIRC: Kidney renal clear cell carcinoma; LIHC: Liver hepatocellular carcinoma; LUAD: Lung adenocarcinoma; LUSC: Lung squamous cell carcinoma; STAD: Stomach adenocarcinoma; STES: Stomach and Esophageal carcinoma; THCA: Thyroid carcinoma; UCEC: Uterine Corpus Endometrial Carcinoma.

### DETAILED DESCRIPTION

Testing using cell-free DNA (cfDNA) mainly relies on whole genome sequencing without performing any prior experimental enrichment of cfDNA molecules from clinically-relevant DNA (e.g., from fetal or tumor tissues). We envision that there is still room to improve the performance of disease detection and tissue-of-origin analysis (e.g., higher specificity and sensitivity) and cost-effectiveness for actual clinical applications, if one could selectively analyze the subset of cfDNA molecules that enrich the contribution of the target tissue of interest.

Some previous studies used approaches based on DNA immunoprecipitation or hybridization capture to enrich the tissue-specific molecules according to the markers showing differential methylation patterns at CpG sites between tissues (Shen et al. Nature. 2018; 563:579-583; Liu et al. Ann Oncol. 2020; 31:745-759). DNA immunoprecipitation-based approaches have limitations in their application, such as the related longer turnover time and variability between different antibody vendor production lots (Shen et al. Nat Protoc. 2019; 14:2749-2780). Those technologies involving bisulfite conversion often result in DNA degradation and quantities of DNA loss. For example, the bisulfite treatment would lead to less than 20% average recovery of DNA (Kresse et al. Clin Epigenet. 2023; 15:151).

In this disclosure, we propose using fragmentomic features for enriching the tissue-specific signals, which may provide a new avenue to explore the enrichment of targeted tissue signals in liquid biopsy. These fragmentomic features can include end motifs (e.g., resulting from methylation-associated cutting positions) and fragment sizes. Methylation-associated cutting positions can be the genomic positions corresponding to the recurrent cutting ends of cfDNA molecules derived from one or more tissues. The end motif can refer to the compositions of multiple nucleotides close to the end of a cfDNA molecule, namely k-mer end motifs (‘k’ represents the number of ending nucleotides of interest). The value of ‘k’ can be but not limited to 1, 2, 3, 4, 5, 6, 7, 8, 9, 10, etc. The fragment size refers to the total number of nucleotides in a cfDNA fragment. The fragment size can be determined by the outmost genomic coordinates of a pair of paired-end reads that are aligned to a reference genome. The fragment size can also be determined by sequencing an entire fragment with a longer read length (e.g., long-read sequencing technologies).

One advantage of fragmentomic features is that it can capture a broader range of molecular information in a single assay, including but not limited to nuclease activity, DNA methylation signals, and chromatin structures, thus providing the opportunity to improve the performance in disease detection. Additionally, in one example, one can use end-specific polymerase chain reaction (PCR) to effectively enrich the targeted set of cfDNA molecules for downstream analysis, e.g., prior to sequencing.

Accordingly, we developed new approaches for enriching cfDNA molecules derived from a particular cell origin for a cell-free biological sample (e.g., plasma, serum, urine, saliva) based on one or more end-specific signatures of cfDNA. For example, one may use CG-containing end motifs, which may be further differentially presented in the tissue-specific methylation regions, to mediate PCR reaction, followed by sequencing. Other types of fragmentomic features can also be used alone or in combination for this purpose, e.g., cutting position, fragment end motifs, and fragment sizes. In various embodiments, the enrichment steps can be enabled using targeted capture sequencing, quantitative polymerase chain reaction (qPCR), digital PCR (dPCR), or droplet digital PCR (ddPCR).

### I. Cell-Free DNA End Motifs and CPG Sites

After cutting by a nuclease, cfDNA fragments have ending sequences at both ends of the fragment. The specific ending sequences can be analyzed as end motifs (certain ending sequences) that have certain properties (e.g., a given type of end motif) can be enriched for clinically-relevant DNA. Example types of end motifs are CGN and NCG, which are discussed in more detail below.

### A. End Motifs

An end motif relates to the ending sequence of a cell-free DNA fragment, e.g., the sequence for the K bases at either end of the fragment. The ending sequence can be a k-mer having various numbers of bases, e.g., 1, 2, 3, 4, 5, 6, 7, etc. The end motif (or “sequence motif”) relates to the sequence itself as opposed to a particular position in a reference genome. Thus, a same end motif may occur at numerous positions throughout a reference genome. The end motif may be determined using a reference genome, e.g., to identify bases just before a start position or just after an end position. Such bases will still correspond to ends of cell-free DNA fragments, e.g., as they are identified based on the ending sequences of the fragments.

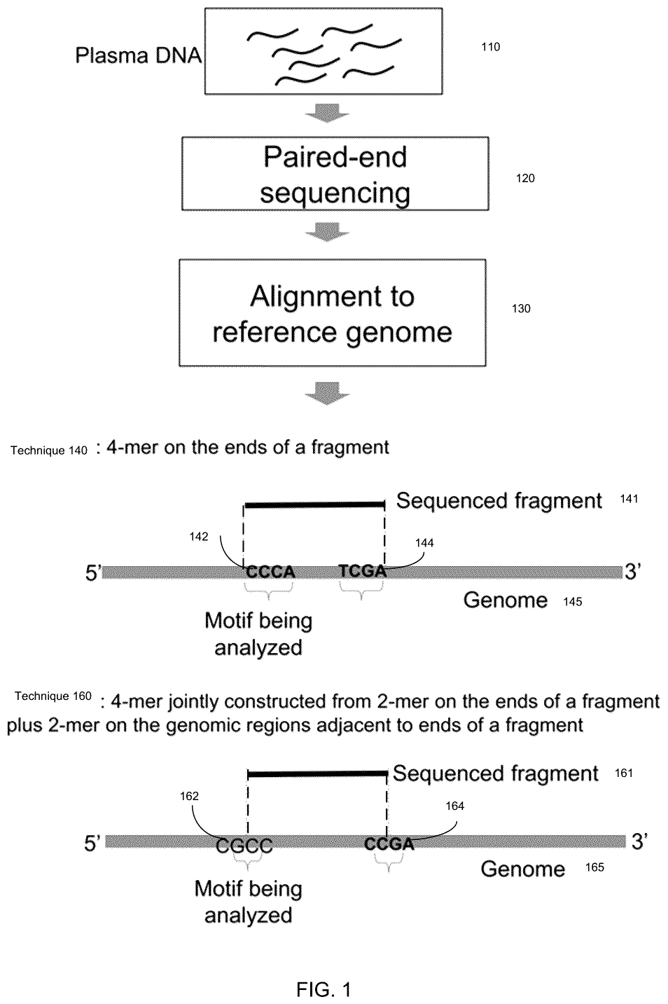

FIG. 1 shows examples for end motifs according to embodiments of the present disclosure. FIG. 1 depicts two ways to define 4-mer end motifs to be analyzed. In technique 140, the 4-mer end motifs are directly constructed from the first 4-bp sequence on each end of a cfDNA molecule (e.g., from plasma or urine). For example, the first 4 nucleotides or the last 4 nucleotides of a sequenced fragment could be used. In technique 160, the 4-mer end motifs are jointly constructed by making use of the 2-mer sequence from the sequenced ends of fragments and the other 2-mer sequence from the genomic regions adjacent to the ends of that fragment. In other embodiments, other types of motifs can be used, e.g., 1-mer, 2-mer, 3-mer, 5-mer, 6-mer, and 7-mer end motifs.

As shown in FIG. 1, cell-free DNA fragments 110 are obtained, e.g., using a purification process on a blood sample, such as by centrifuging. Besides plasma DNA fragments, other types of cell-free DNA molecules can be used, e.g., from serum, urine, saliva, and other such cell-free samples mentions herein. In one embodiment, the DNA fragments may be blunt-ended.

At block 120, the DNA fragments are subjected to paired-end sequencing. In some embodiments, the paired-end sequencing can produce two sequence reads from the two ends of a DNA fragment, e.g., 30-120 bases per sequence read. These two sequence reads can form a pair of reads for the DNA fragment (molecule), where each sequence read includes an ending sequence of a respective end of the DNA fragment. In other embodiments, the entire DNA fragment can be sequenced, thereby providing a single sequence read, which includes the ending sequences of both ends of the DNA fragment.

At block 130, the sequence reads can be aligned to a reference genome. This alignment is to illustrate different ways to define a sequence motif and may not be used in some embodiments. The alignment procedure can be performed using various software packages, such as BLAST, FASTA, Bowtie, BWA, BFAST, SHRiMP, SSAHA2, NovoAlign and SOAP.

Technique 140 shows a sequence read of a sequenced fragment 141, with an alignment to a genome 145. With the 5′ end viewed as the start, a first end motif 142 (CCCA) is at the start of sequenced fragment 141. A second end motif 144 (TCGA) is at the tail of the sequenced fragment 141.

Technique 160 shows a sequence read of a sequenced fragment 161, with an alignment to a genome 165. With the 5′ end viewed as the start, a first end motif 162 (CGCC) has a first portion (CG) that occurs just before the start of sequenced fragment 161 and a second portion (CC) that is part of the ending sequence for the start of sequenced fragment 161. A second end motif 164 (CCGA) has a first portion (GA) that occurs just after the tail of sequenced fragment 161 and a second portion (CC) that is part of the ending sequence for the tail of sequenced fragment 161. For technique 160, the number of bases from the adjacent genome regions and sequenced plasma DNA fragments can be varied and are not necessarily restricted to a fixed ratio, e.g., instead of 2:2, the ratio can be 2:3, 3:2, 4:4, 2:4, etc.

As the ending sequence is used to align the sequence read to the reference genome, any sequence motif determined from the ending sequence or just before/after is still determined from the ending sequence. Thus, technique 160 makes an association of an ending sequence to other bases, where the reference is used as a mechanism to make that association. A difference between techniques 140 and 160 would be to which two end motif a particular DNA fragment is assigned, which affects the particular values for the relative frequencies of different end motifs. But the overall result (e.g., fractional concentration of clinically-relevant DNA, classification of a level of pathology, etc.) would not be affected by how the DNA fragment is assigned to an end motif, as long as a consistent technique is used for the training data as used in production.

### B. Cutting Positions Around CpG Sites

Certain end motifs may occur around CpG sites depending on the cutting position. For example, the CG may occur at the end of the fragment, resulting in a CG end motif, also referred to as a CGN end motif, where N is any base. As another example, the CG may occur at the second position from the end of the fragment, resulting in an NCG end motif.

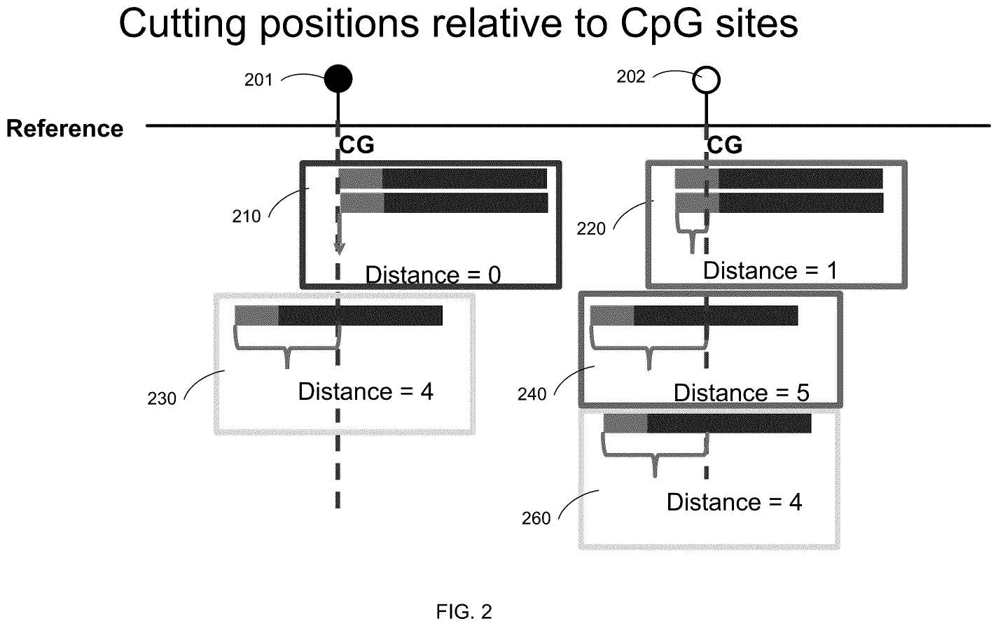

FIG. 2 illustrates cutting positions relative to CpG sites (also referred to as CG sites) according to embodiments of the present disclosure. The horizontal line refers to a reference sequence, e.g., part of a reference genome, which contains two CpG sites. After sequencing, the resulting reads can be mapped to this region. The distance between the fragment ends and the position relative to the CpG sites can be calculated. For example, the fragments 210 ended exactly at the CG position 201, and thus have a distance of 0 (also referred to as position 0).

The fragments 220 end one position to the left of CpG site 202. Since the fragment was cut one base before the CpG site, the distance is considered one. Similarly, since the fragments 230 end four positions to the left of CpG site 201, the distance is considered four. Since the fragments 240 and 260 end five positions and four positions to the left of CpG site 202, their respectively distances are considered five and four, respectively. In this manner, we can calculate the distance between the cutting ends and the CpG sites and group the fragments with the same distance together.

FIG. 2 shows, as illustrated by boxes of different colors, how DNA fragments can be grouped depending on distance from the 5′ ends to the CpG site. If the first two nucleotides at the 5′ end of a fragment are CG, the aforementioned distance is 0. If there is one nucleotide at the 5′ end immediately preceding the CG, the aforementioned distance is 1, which corresponds to fragments having an NCG motif, where N is any one of the 4 bases. Accordingly, since fragments 230 and 260 both have a distance of four from the CpG sites 201 and 202, fragments 230 and 260 may be included in the same group based on their distances from the 5′ ends to the CpG site(s).

Accordingly, an end of the cell-free DNA molecule has a first position at an outermost position, a second position that is next to the first position, and a third position that is next to the second position. Fragments 210 can be considered to have CGN end motifs that have C at the first position and G at the second position. Fragments 220 can be considered to have NCG end motifs that have C at the second position and G at the third position. Cell-free DNA fragments carrying a cutting position one base before the CpG site (i.e., carrying NCG motif) or at the CpG site (i.e., carrying CGN end motif) can be selected for downstream analysis, e.g., fetal or tumor DNA fraction quantification.

### II. Enrichment of Clinically-Relevant DNA Using NCG Motifs

To investigate if we can enrich clinically-relevant DNA through its methylation-associated fragmentomic features, cfDNA fragments carrying CpG-containing motifs were analyzed. As shown below, cfDNA fragments with NCG end motifs have an increased tissue fraction (e.g., fetal/tumor fraction) for clinically-relevant DNA.

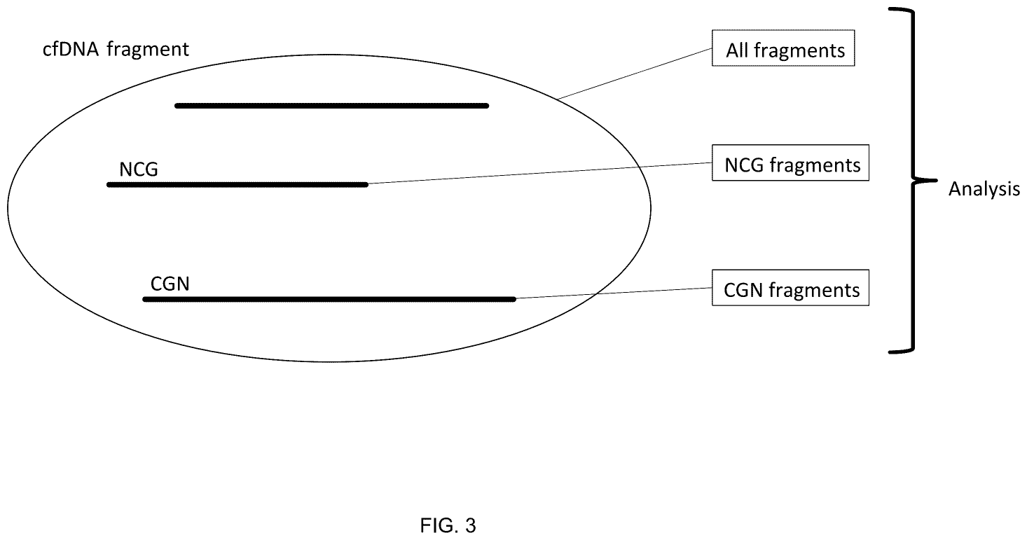

FIG. 3 shows different types of cfDNA fragments, including NCG fragments and CGN fragments, which can be analyzed to determine a property of a biological sample or of a subject from which the sample was obtained. Such a property can be of clinically-relevant DNA. The biological sample can include clinically-relevant DNA and other DNA that are cell-free in the sample. As examples, the cfDNA may be fetal DNA fragments, tumor DNA fragments, etc. The CGN and NCG DNA fragments may be analyzed separately as a group. Thus, embodiments can include analyzing a particular group of cell-free DNA molecules to determine a property of the clinically-relevant DNA the biological sample.

### A. Fetal Example

Methylation-associated cutting positions can be used to enrich cfDNA of fetal origin. The placental genome is globally hypomethylated in comparison with the blood cells, resulting in the higher probability of cfDNA cleavage at the 1-nt position before CpG sites, generating relatively more molecules carrying NCG motifs but relatively fewer molecules carrying CGN motifs. Hence, the enrichment of molecules with NCG motifs would enrich the fetal DNA, whereas the enrichment of molecules with CGN motifs would deplete the fetal DNA.

To test this hypothesis, we analyzed the fetal DNA fraction of all cfDNA fragments, cfDNA fragments carrying NCG end motif, and cfDNA fragments carrying CGN end motif in 30 pregnancy samples from different trimester (median paired-end sequencing reads: 206 million (IQR: 142-232 million)). The genotypes regarding the maternal buffy coat and placenta/chorionic villus tissue samples were obtained using microarray-based genotyping technology (HumanOmni2.5 genotyping array Illumina), and informative SNPs were identified (i.e., where the mother was homozygous (denoted as AA genotype), and the fetus was heterozygous (denoted as AB genotype)). Fetal-specific DNA fragments were identified according to the DNA fragments carrying fetal-specific alleles at informative SNP sites. In this scenario, the B allele was fetal-specific, and the DNA fragments carrying the B allele were deduced to be originated from fetal tissues. The number of fetal-specific molecules (p) carrying the fetal-specific alleles (B) was determined. The number of molecules (q) carrying the shared alleles (A) was determined. The fetal DNA fraction across all cell-free DNA samples was calculated by 2p/(p+q)*100%.

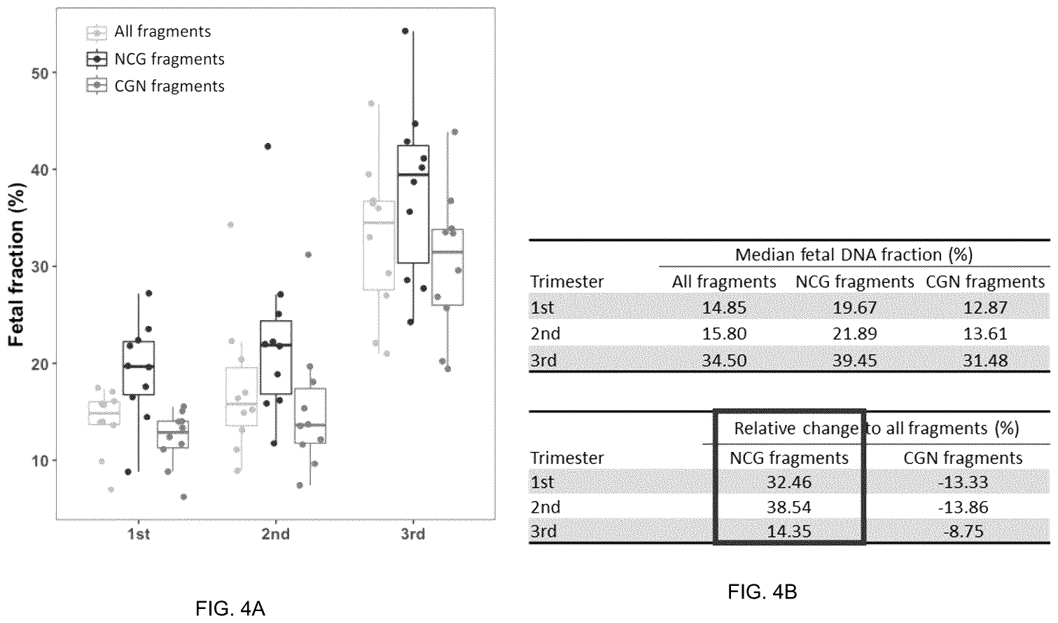

FIGS. 4A-4B show the fetal fractional concentration of NCG-ended DNA fragments, CGN-ended DNA fragments, and all DNA fragments. Compared with all fragments, NCG-ended DNA fragments show an increased fetal fraction across the first trimester, the second trimester and the third trimester.

As shown in FIGS. 4A-B, selective analysis of cfDNA fragments carrying NCG motif showed around 32.46%, 38.54%, and 14.35% increase of fetal DNA fraction in 1st (median fetal fraction: NCG fragments: 19.67% vs. all fragments: 14.85%), 2nd (median fetal fraction: NCG fragments: 21.89% vs. all fragments: 15.80%), and 3rd (median fetal fraction: NCG fragments: 39.45% vs. all fragments: 34.50%) trimester, respectively.

Selective analysis of cfDNA fragments carrying CGN motif showed around 13.33%, 13.86%, and 8.75% decrease of fetal DNA fraction in 1st (median fetal fraction: CGN fragments: 12.87% vs. all fragments: 14.85%), 2nd (median fetal fraction: CGN fragments: 13.61% vs. all fragments: 15.80%), and 3rd (median fetal fraction: CGN fragments: 31.48% vs. all fragments: 39.45%) trimester, respectively.

These results suggested the selective analysis of a subset of NCG-ended cfDNA molecules could enrich the targeted molecules originating from a particular organ (e.g., placenta). The fetal DNA fraction is a crucial parameter determining the overall performance of NIPT. The higher the fetal DNA fraction, the higher the performance (e.g., accuracy, amount of sample required, amount of reagents, etc.) the NIPT test could achieve. The higher the fetal DNA fraction, the fewer the sequence reads would be required to achieve the desired performance. Thus, the enrichment of fetal DNA fraction through the selective analysis of a subset of cfDNA molecules would be valuable for clinical and commercial reasons. One could use this cost-effective approach without adversely affecting the performance, even with better performance, to test the pregnant subjects with initially low fetal DNA fraction.

### B. Tumor Example

As a further example, methylation-associated cutting positions can be used to enrich cfDNA of tumoral origin. In general, the tumoral genome is globally hypomethylated in comparison with the blood cells, resulting in a higher probability of cfDNA cleavage at the 1-nt position before CpG sites, generating relatively more molecules carrying NCG motifs but relatively fewer molecules carrying CGN motifs. Hence, the enrichment of molecules with NCG motifs would enrich the tumor DNA, whereas the enrichment of molecules with CGN motifs would deplete the tumor DNA.

To test it, we analyzed the tumor DNA fraction of all cfDNA fragments, cfDNA fragments carrying NCG end motif, and cfDNA fragments carrying CGN end motif in one hepatocellular carcinoma case (HCC; paired-end sequencing reads: 4,943 million). HCC is used merely as an example, as embodiments apply to other types of cancer as well.

HCC-specific mutations were obtained using whole-genome sequencing of buffy coat (paired-end sequencing reads: 1,310 million) and HCC tissue (paired-end sequencing reads: 1,165 million). HCC-specific mutations were defined as mutations identified in the HCC tissue but not in the buffy coat. HCC-specific DNA fragments were identified according to the DNA fragments carrying HCC-specific mutations. The number of HCC-specific molecules (p) carrying the HCC-specific mutant alleles was determined. The number of molecules (q) carrying wide-type alleles was determined. The tumor DNA fraction across all cell-free DNA samples would be calculated by p/(p+q)*100%.

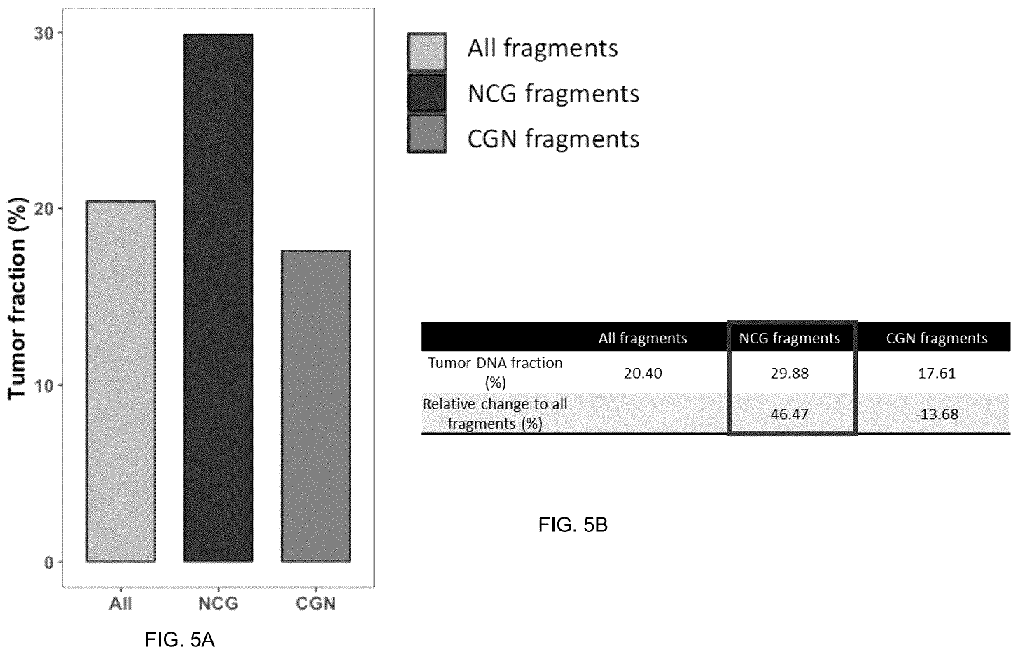

FIGS. 5A-5B show the tumor fractional concentration of NCG-ended DNA fragments, CGN-ended DNA fragments, and all fragments tumor DNA. As shown in FIG. 5A-B, selective analysis of cfDNA fragments carrying NCG motif showed around 46.47% increase in tumor DNA fraction (tumor fraction: NCG fragments: 29.88% vs. all fragments: 20.40%), and selective analysis of cfDNA fragments carrying CGN motif showed around 13.68% decrease of tumor DNA fraction (tumor fraction: CGN fragments: 17.61% vs. all fragments: 20.40%). These results suggested the selective analysis of a subset of NCG-ended cfDNA molecules could indeed enrich the targeted molecules originating from tumor cells, thus potentially offering a more sensitive approach for cancer detection. The benefits described above for an increased fetal fraction also apply to an increased tumor fraction.

### III. Enrichment Using Tissue-Specific Methylation

Cell-free DNA cleavages on CpG sites can depend on methylation states. There is a higher probability of cfDNA cutting occurring at the methylated CpG sites than at the unmethylated CpG sites. Hence, if one selects the cfDNA molecules containing the CGN end motif from tissue-specific methylation regions, those selected cfDNA molecules would enrich the molecules derived from that tissue. The targeted tissue is also referred to as clinically-relevant DNA. The tissue-specific methylation regions can be determined beforehand from previous publications, such as bisulfite sequencing results or other single-molecule real-time sequencing results (nanopore sequencing or PacBio SMRT sequencing). Conversely, if one focuses on the tissue-specific unmethylation regions, that would confer a relatively higher frequency of cfDNA cutting at the position immediately before the CpG site. In that case, the enrichment of cfDNA molecules carrying NCG may be informative for those hypomethylation regions.

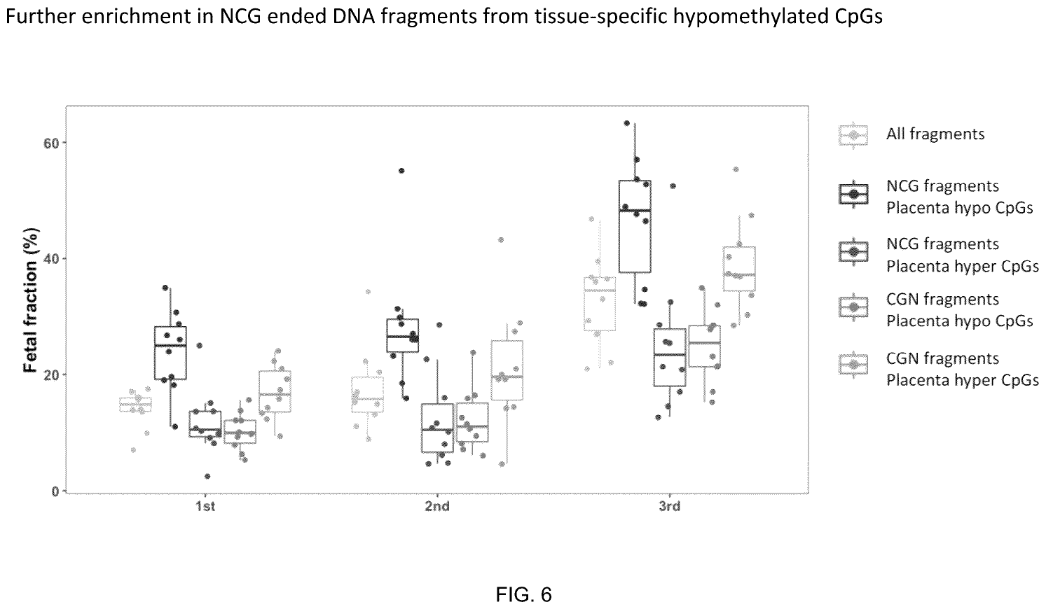

FIG. 6 shows different types of cfDNA fragments, including NCG fragments and CGN fragments, which can be analyzed at tissue-specific methylated CpG sites to determine a property of a biological sample or of a subject from which the sample was obtained. Cell-free DNA fragments carrying NCG or CGN end motifs can be used. These fragments can be further selected based on the methylation status of the CpG site located at the end motif. For example, cfDNA fragments carrying tissue-specific hypermethylated and hypomethylated NCG end motif and cfDNA fragments carrying tissue-specific hypermethylated and hypomethylated CGN end motif can be selected for downstream analysis, e.g., fetal DNA fraction or tumor DNA fraction quantification.

Enrichment of a subset of clinically-relevant cfDNA can be performed bioinformatically or experimentally by various embodiments described in this disclosure. For example, we can use PCR with the first primer targeting CGN end motifs and the second primer targeting the tissue-specific methylation regions, thus enriching the targeted cfDNA molecules derived from that tissue.

Accordingly, in contrast to making the genome-wide global methylation pattern, one could perform the end-motif based selective analysis of cfDNA molecules derived from tissue-specific methylation markers. The enhanced enrichment of cfDNA derived from the targeted tissue could be achieved by adjusting the criteria for the strength of methylation difference for those markers between the targeted tissue and other tissues (i.e., the tissue-specific methylation regions). For example, one could enhance the enrichment of clinically-relevant DNA by using the methylation-associated cutting positions in those markers showing the methylation difference at least but not limited to 5%, 10%, 20%, 30%, 40%, 50%, 60%, 70%, 90%, etc.

### A. Fetal Example

As proposed above, the selective analysis of cfDNA carrying the placenta-specific hypomethylated NCG and/or the placenta-specific hypermethylated CGN can enrich the fetal DNA fraction. For comparison purposes, we determined the fetal DNA fractions for all cfDNA fragments and cfDNA fragments carrying methylation-configured end motifs in 30 pregnancy samples from different trimesters, respectively. The methylation-configured end motifs included the placenta-specific hypomethylated NCG and CGN and the placenta-specific hypermethylated NCG and CGN end motifs. Placenta-specific hypermethylated and hypomethylated CpG sites were identified using bisulfite sequencing results of the buffy coat (sequencing depth: 75× haploid genome coverage) and the placenta tissues (sequencing depth: 82× haploid genome coverage). Placenta-specific hypermethylated sites were defined by those CpG sites with a methylation density of over 70% in the placenta tissues but below 30% in the buffy coat samples. Placenta-specific hypomethylated sites were defined by those CpG sites with a methylation density of below 30% in the placenta tissues but over 70% in the buffy coat samples, although also percentages can be used, e.g., as mentioned herein.

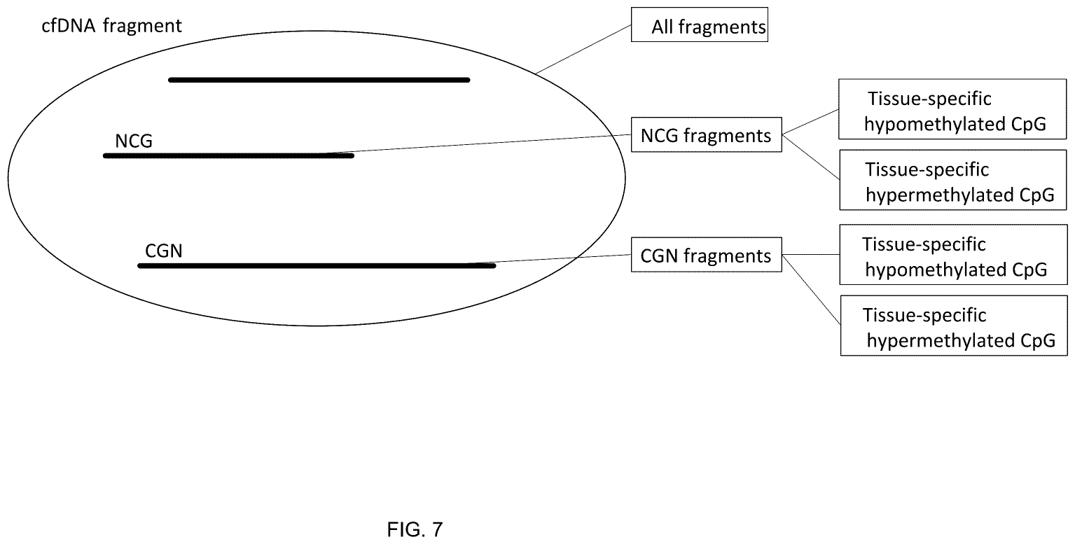

FIG. 7 shows boxplots illustrating the fractional concentration of NCG DNA fragments (placenta hypomethylated CpGs), NCG DNA fragments (placenta hypermethylated CpGs), CGN DNA fragments (placenta hypomethylated CpGs), CGN DNA fragments (placenta hypermethylated CpGs), and all DNA fragments (placenta hypomethylated CpGs). Compared with the fractional concentration of fetal DNA as illustrated in FIGS. 4A-4B, these results show even more improved increase of fetal DNA fractional concentration for NCG DNA fragments at the placenta hypomethylated CpGs. Further, there is an increase for CGN DNA fragments at the placenta hypermethylated sites.

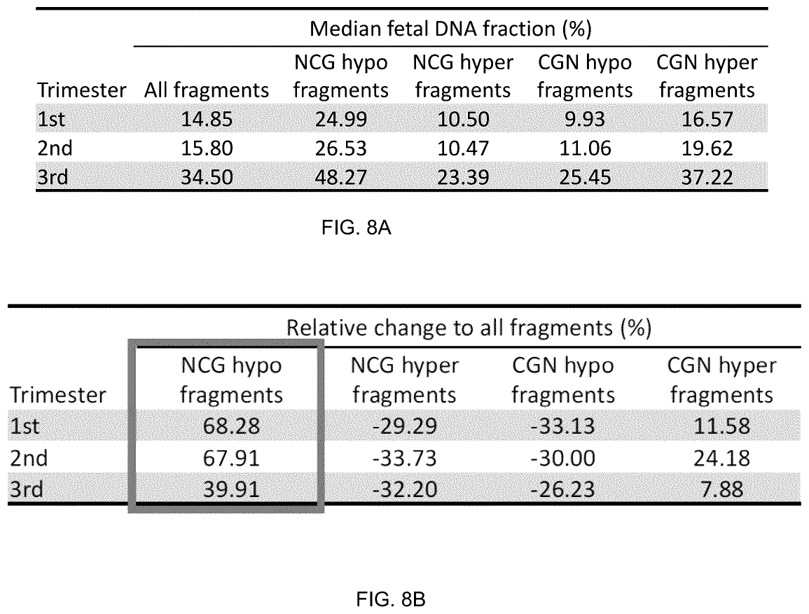

FIG. 8A-8B show tables of the fractional concentration of NCG DNA fragments (placenta hypomethylated CpGs), NCG DNA fragments (placenta hypermethylated CpGs), CGN DNA fragments (placenta hypomethylated CpGs), CGN DNA fragments (placenta hypermethylated CpGs), and all DNA fragments.

As shown in these figures, the selective analysis of cfDNA fragments carrying the placenta-specific hypermethylated CGN and hypomethylated NCG end motifs indeed gave the relative enrichment in fetal DNA fraction, whereas those carrying the placenta-specific hypermethylated NCG and hypomethylated CGN end motif did not. In particular, the selective analysis of cfDNA fragments carrying placenta-specific hypomethylated NCG motif gave the most enrichment, showing around 68.28%, 67.91%, and 39.91% increase of fetal DNA fraction in plasma of those pregnant women in the 1st (median fetal fraction: NCG fragments: 24.99% vs. all fragments: 14.85%), 2nd (median fetal fraction: NCG fragments: 26.53% vs. all fragments: 15.80%), and 3rd (median fetal fraction: NCG fragments: 48.27% vs. all fragments: 34.50%) trimesters, respectively.

Compared with the enrichment method based on the global hypomethylation (FIG. 4B), there were 27.05%, 22.57%, and 22.36% increases in fetal DNA fraction using the placenta-specific methylation markers for those pregnant women in the 1st (median fetal fraction: cfDNA fragments carrying the placenta-specific hypomethylated NCG end motifs: 24.99% vs. all cfDNA fragments carrying NCG end motifs: 19.67%), 2nd (median fetal fraction: cfDNA fragments carrying the placenta-specific hypomethylated NCG end motifs: 26.53% vs. all cfDNA fragments carrying NCG end motifs: 21.89%), and 3rd (median fetal fraction: cfDNA fragments carrying the placenta-specific hypomethylated NCG end motifs: 48.27% vs. all cfDNA fragments carrying NCG end motifs: 39.48%) trimesters, respectively (FIG. 4B and FIG. 8A). The hypomethylated and hypermethylated placenta-specific markers showed an average of 65.11% methylation difference between the placenta and the white blood cells, which was greater than the global methylation difference with an average of 20.48%.

The data indicates that enhanced enrichment of fetal DNA fraction could be achieved by the selection of cfDNA molecules with targeted end motifs that are associated with a set of genomic regions exhibiting higher tissue specificity in terms of methylation patterns. And more specifically, by analyzing NCG DNA fragments from placenta-specific hypomethylated CpG sites, a dramatically increased fractional concentration of the fetal DNA can be achieved. And more specifically, by analyzing CGN DNA fragments from placenta-specific hypermethylated CpG sites, an increased fractional concentration of the fetal DNA can be achieved.

### B. Tumor Example

As another example, the selective analysis of cfDNA carrying the tumor-specific hypomethylated NCG and/or the tumor-specific hypermethylated CGN can be used to enrich tumor DNA fraction. For comparison purposes, we determined the tumor DNA fractions for all cfDNA fragments and cfDNA fragments carrying methylation-configured end motifs, respectively. The methylation-configured end motifs included the tumor-specific hypermethylated NCG and CGN and the tumor-specific hypermethylated NCG and CGN end motifs. HCC-specific hypermethylated and hypomethylated CpG sites were identified using bisulfite sequencing results of the buffy coat (sequencing depth: 75× haploid genome coverage) and HCC tissues (sequencing depth: 21× haploid genome coverage). HCC-specific hypermethylated sites were defined by those CpG sites with a methylation density of over 70% in the HCC tissues but below 30% in the buffy coat samples. HCC-specific hypomethylated sites were defined by those CpG sites with a methylation density of below 30% in the HCC tissues but over 70% in the buffy coat samples, although also percentages can be used, e.g., as mentioned herein.

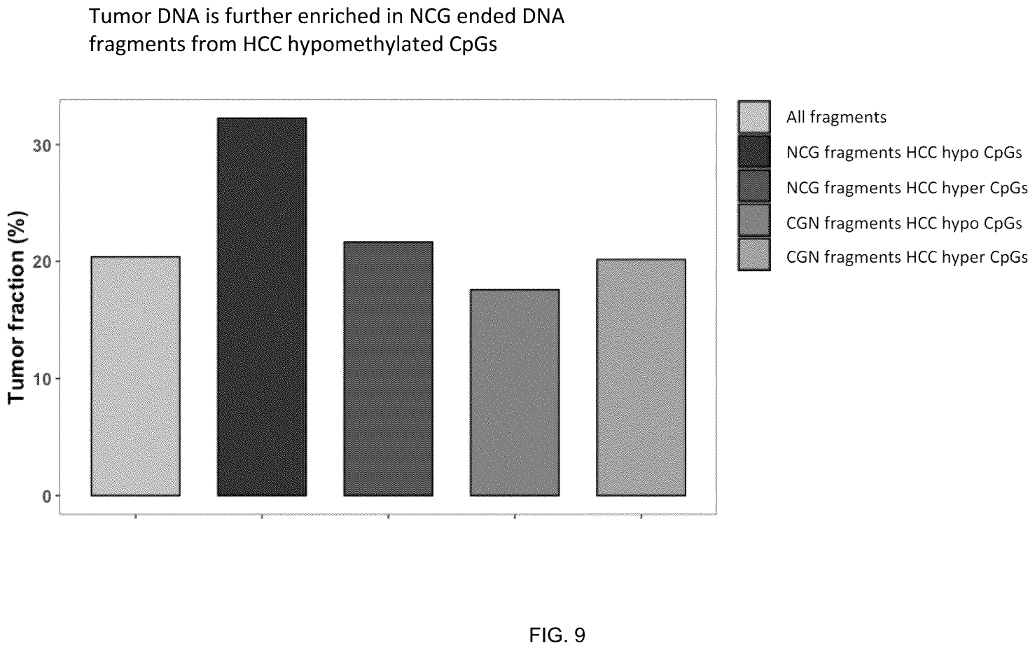

FIG. 9 shows the fractional concentration of NCG DNA fragments (HCC hypomethylated CpGs), NCG DNA fragments (HCC hypermethylated CpGs), CGN DNA fragments (HCC hypomethylated CpGs), CGN DNA fragments (HCC hypermethylated CpGs), and all DNA fragments. As illustrated by FIG. 9, compared with all DNA fragments, the fractional concentrations of NCG DNA fragments (HCC hypomethylated CpGs) and the NCG fragments (HCC hypermethylated CpGs) tumor DNA have been increased.

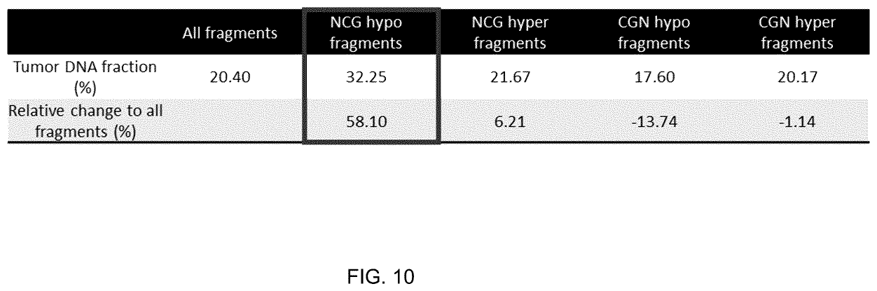

FIG. 10 shows the tumor fractional concentration in NCG DNA fragments (HCC hypomethylated CpGs), NCG DNA fragments (HCC hypermethylated CpGs), CGN DNA fragments (HCC hypomethylated CpGs), CGN DNA fragments (HCC hypermethylated CpGs), and all DNA fragments tumor.

As illustrated by FIG. 10, compared with all DNA fragments, the fractional concentration of NCG DNA fragments (HCC hypomethylated CpGs) is increased from 20.4% to 32.25%, which corresponds to a 58.10% relative change to all fragments tumor DNA. Additionally, compared with all DNA fragments, the fractional concentration of NCG DNA fragments (HCC hypermethylated CpGs) increased from 2.4% to 21.67%, which corresponds to a 6.21% relative change to all fragments tumor DNA.

As shown in these figures, the selective analysis of cfDNA fragments carrying the tumor-specific hypomethylated NCG end motifs indeed gave the relative enrichment in tumor DNA fraction. Accordingly, these increased fractional concentrations further indicated that an enrichment of tumor DNA can be achieved by using methods disclosed herein.

As described above, the selective analysis of cfDNA fragments carrying HCC-specific hypomethylated NCG motif gave the most enrichment, showing around 58.10% increase in tumor DNA fraction (median tumor fraction: HCC-specific hypomethylated NCG fragments: 32.25% vs. all fragments: 20.40%). Compared with the enrichment method based on the global hypomethylation, there was a 7.93% increase in tumor DNA fraction for cfDNA fragments carrying HCC-specific hypomethylated NCG motif. Those markers showed a median of 63.47% methylation difference between the HCC tumor tissues and the white blood cells, which was greater than the global methylation difference with a median of 20.37%. The data suggested that the enhanced enrichment of tumor DNA fraction could be achieved by the selection of cfDNA molecules with targeted end motifs that are associated with a set of genomic regions exhibiting higher tissue specificity in terms of methylation patterns.

### IV. Enrichment Using Specific 3-Mer End Motifs

As discussed above, by analyzing NCG fragments carrying placenta-specific hypomethylated CpG site, dramatically increased fractional concentration of the fetal DNA can be achieved. As “N” of the NCG and CGN fragments may be any one of the 4 bases (A, C, G, T), the NCG (placenta hypomethylated CpGs) fragments can be further separated into subgroups (i.e., based on the respective “N” base and corresponding types of end motif). Specifically, the DNA fragments may be separated into ACG subgroup, CCG subgroup, GCG subgroup, and TCG subgroup.

Accordingly, we further tested whether a better enrichment of clinically-relevant DNA could be made by the synergetic use of its methylation-associated cutting positions, methylation status, and end motifs. A series of selection steps of cfDNA can be applied to enrich the tissue-specific cfDNA molecules based on methylation patterns and end motifs.

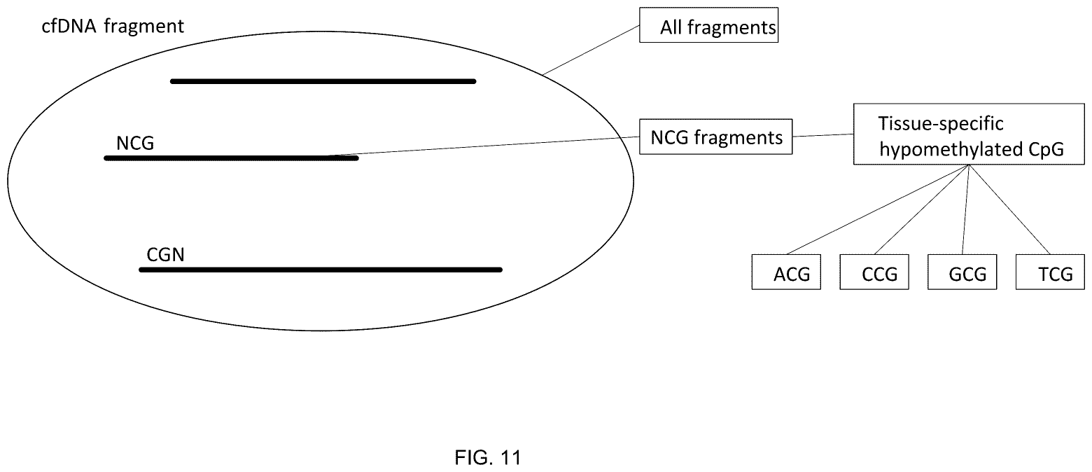

FIG. 11 shows a series of selection steps, including NCG fragments separately having ACG, CCG, GCG, and TCG fragments, which were then analyzed at tissue-specific methylated CpG sites to enrich a sample for clinically-relevant DNA. For example, as shown in FIG. 11, the first step may be to select the cfDNA fragments carrying NCG end motifs. A second step may be to select those fragments obtained in the first step according to methylation patterns fulfilling a certain criterion. The criteria can be the methylation difference between the targeted tissue and other tissues of at least but not limited to 5%, 10%, 20%, 30%, 40%, 50%, 60%, 70%, 90%, etc. As shown the difference indicates hypomethylation, but hypermethylation could also be selected or selected instead.

A third step may be to select those fragments obtained in the second step according to the base compositions in CG-containing motifs. For example, cfDNA fragments carrying 5′ ACG, TCG, CCG, and GCG end motifs derived from a tissue-specific hypomethylated CpG site would be used for the downstream analysis, e.g., fetal DNA fraction or tumor DNA fraction quantification. The aforementioned steps can be organized in different orders.

### A. Fetal Example

The fetal DNA fractions of all cfDNA fragments and cfDNA fragments carrying placenta-specific hypomethylated ACG, TCG, CCG, and GCG end motifs in 30 pregnancy samples from different trimesters were analyzed. Placenta-specific hypomethylated CpG sites were identified using bisulfite sequencing results of the buffy coat (sequencing depth: 75× haploid genome coverage) and the placenta tissues (sequencing depth: 82× haploid genome coverage). Placenta-specific hypomethylated sites were defined by those CpG sites with a methylation density of below 30% in the placenta tissues but over 70% in the buffy coat samples, although also percentages can be used, e.g., as mentioned herein.

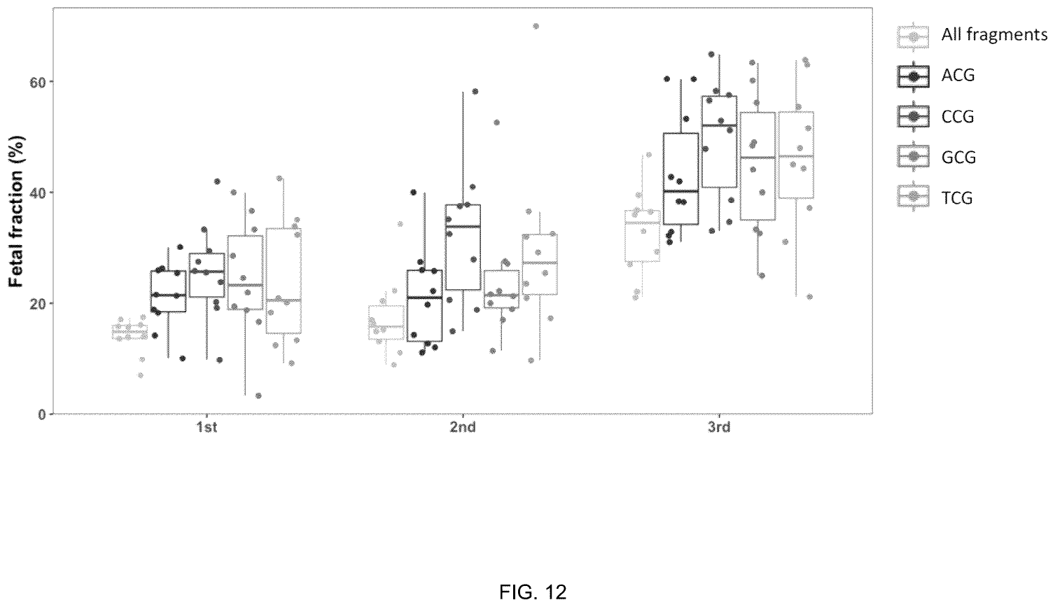

FIG. 12 shows boxplots of the fractional concentration of ACG DNA fragments, CCG DNA fragments, GCG DNA fragments, TCG DNA fragments, and all DNA fragments at placenta hypomethylated CpGs. Compared with the fractional concentration of fetal DNA enriched by tissue-specific methylation as illustrated in FIGS. 7-8B, the results show even more of an increase of fetal DNA fractional concentration, specifically for CCG. The fractional concentrations of the four types of NCG motifs (e.g., ACG, CCG, GCG, and TCG) are increased when compared with the fractional concentration of all DNA fragments fetal DNA. Specifically, across all four types of NCG motifs (e.g., ACG, CCG, GCG, and TCG), CCG appears to have the most improved increase in the fractional concentration of fetal DNA in the first trimester, the second trimester, and the third trimester.

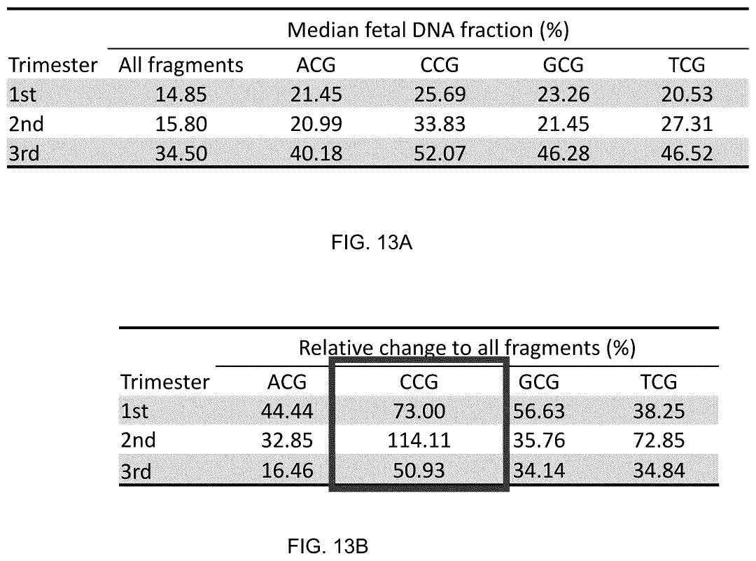

FIGS. 13A-13B show tables of the fractional concentration of ACG DNA fragments, CCG DNA fragments, GCG DNA fragments, TCG DNA fragments, and all DNA fragments at placenta hypomethylated CpGs. The fractional concentrations of the four types of NCG motifs (e.g., ACG, CCG, GCG, and TCG) are increased when compared with the fractional concentration of all DNA fragments. In particular, selective analysis of cfDNA fragments carrying placenta-specific hypomethylated CCG motif showed the highest increase of fetal DNA fraction across all trimesters, which indicated around 73.00%, 114.11%, and 50.93% increase of fetal DNA fraction in 1st (median fetal fraction: placenta-specific hypomethylated CCG fragments: 25.69% vs. all fragments: 14.85%), 2nd (median fetal fraction: placenta-specific hypomethylated CCG fragments: 33.83% vs. all fragments: 15.80%), and 3rd (median fetal fraction: placenta-specific hypomethylated CCG fragments: 52.07% vs. all fragments: 34.50%) trimesters, respectively.

The highest increase of fetal fraction in fragments carrying placenta-specific hypomethylated CCG motif is probably due to the hypomethylation status of the placental genome and a higher DNASE1L3 expression in the placenta tissue. Thus, in some embodiments, one end sequence motif can be used for enrichment, e.g., the one end sequence motif having C at the first position (i.e., CCG).

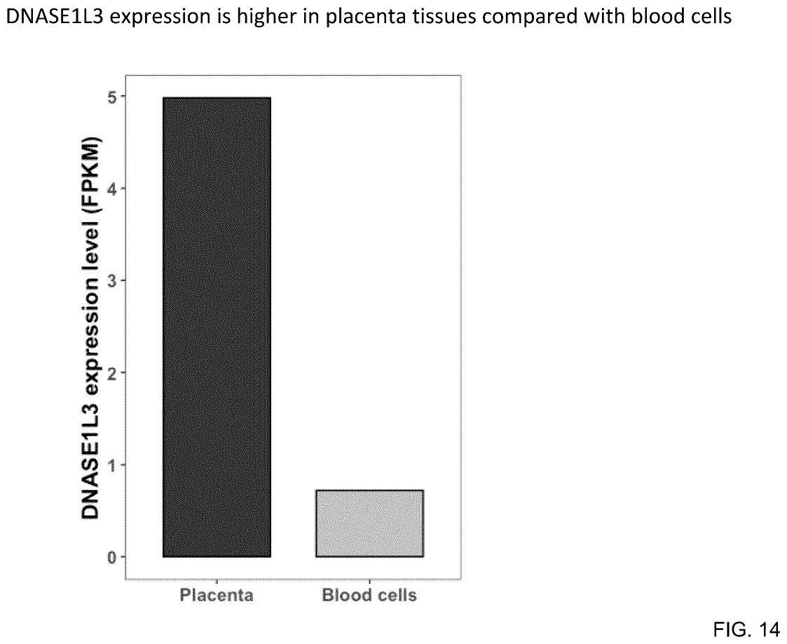

FIG. 14 illustrates the expression level of DNASE1L3 in placenta tissues and blood cells. Since placenta has a higher DNASE1L3 expression level compared with blood cells, CCG DNA fragments (placenta hypomethylated CpGs) tend to have the most increase in the fractional concentration of fetal DNA and thus is a good target for enrichment for clinically-relevant DNA. In other words, the higher hypomethylation rate in fetal DNA would lead to more cutting at NCG site, and the higher DNASE1L3 expression in placenta would lead higher cutting at CC-motif site, which, in turn, lead to a higher cutting probability at the CCG site in the placenta-derived DNA. These data indicated that the use of fragmentomic features to enrich the targeted cfDNA molecules had the advantage of combining the molecular signals from multiple dimensions, potentially improving the diagnostic performance of NIPT.

### B. Tumor Example

As another example, tumor DNA fractions of all cfDNA fragments and cfDNA fragments carrying HCC-specific hypomethylated ACG, TCG, CCG, and GCG end motifs in one HCC case were analyzed. HCC-specific hypomethylated CpG sites were identified using bisulfite sequencing results of the buffy coat (sequencing depth: 75× haploid genome coverage) and HCC tissues (sequencing depth: 21× haploid genome coverage). HCC-specific hypomethylated sites were defined by those CpG sites with a methylation density of below 30% in the HCC tissues but over 70% in the buffy coat samples, although also percentages can be used, e.g., as mentioned herein.

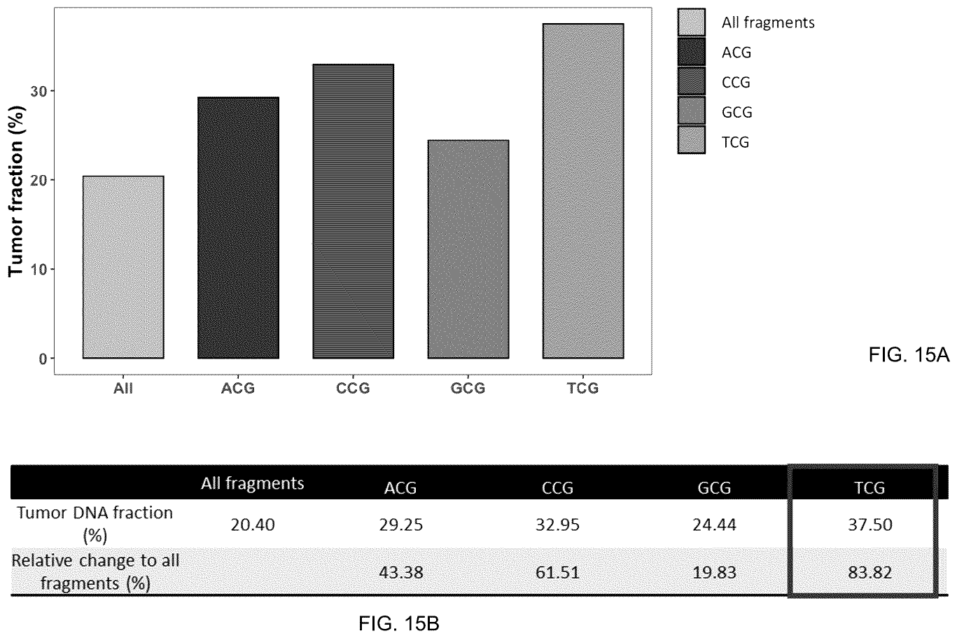

FIGS. 15A-15B show the fractional concentration of ACG DNA fragments, CCG DNA fragments, GCG DNA fragments, TCG DNA fragments, and all DNA fragments at HCC hypomethylated CpGs. As FIG. 15A illustrates, compared with the fractional concentration of tumor DNA enriched by tissue-specific methylation as illustrated in FIGS. 9-10, the results show even more improved increase of tumor DNA fractional concentration, specifically for CCG and especially for TCG.

The fractional concentrations of the four types of NCG motifs (e.g., ACG, CCG, GCG, and TCG) are increased when compared with the fractional concentration of all DNA fragments. Specifically, across all four types of NCG motifs (e.g., ACG, CCG, GCG, and TCG), TCG appears to have the most improved increase in the fractional concentration of tumor DNA. Specifically, the selective analysis of cfDNA fragments carrying HCC-specific hypomethylated TCG motif showed the highest increase of tumor DNA fraction (i.e., around 83.82% increase (median tumor fraction: HCC-specific hypomethylated CCG fragments: 37.50% vs. all fragments: 20.40%)). Thus, the fractional concentration of tumor DNA may be significantly enriched by focusing the analysis over TCG fragments (HCC hypomethylated CpGs) tumor DNA.

The highest increase of tumor fraction in fragments carrying HCC-specific hypomethylated TCG motif is probably due to the hypomethylation status of HCC genome and a lower DNASE1L3 but higher DNASE1 expression in HCC tissue. The higher hypomethylation rate in HCC origin DNA would lead to more cutting at NCG site, and the lower DNASE1L3 and higher DNASE1 expression in HCC would lead higher cutting at T end motif site, which, in turn, lead to high cutting at TCG site in HCC DNA.

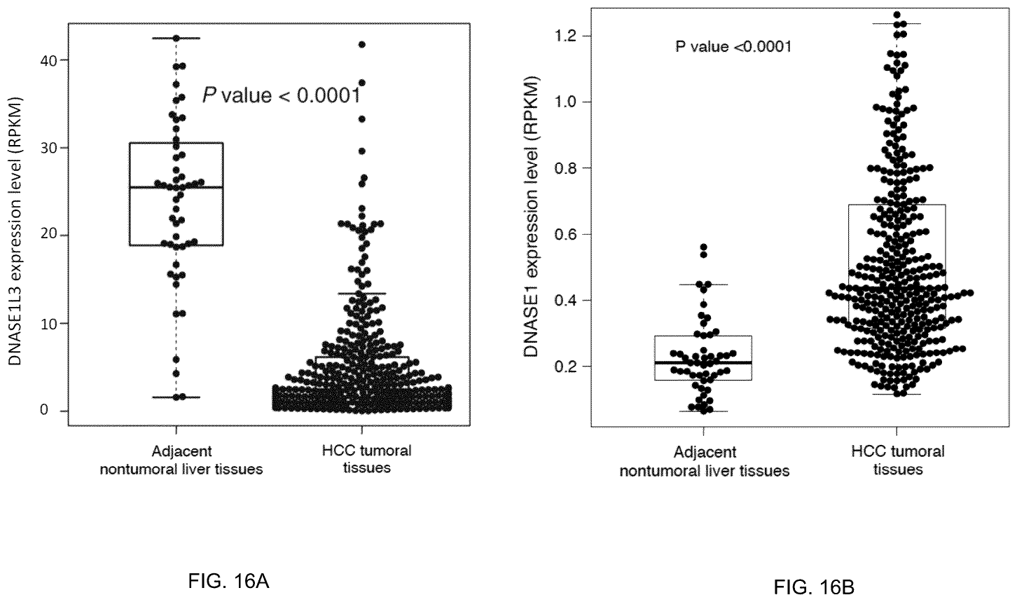

FIG. 16A illustrates the expression level of DNASE1L3 in HCC tumoral tissues and adjacent nontumoral liver tissues. HCC tumoral tissues have a decreased expression level of DNASE1L3 compared to the adjacent nontumoral liver tissues.

FIG. 16B illustrates the expression level of DNASE1 in HCC tumoral tissues and adjacent nontumoral liver tissues. HCC tumoral tissues have an increased expression level of DNASE1 compared to the adjacent nontumoral liver tissues. The increased expression level of DNASE1 leads to the most increase in the fractional concentration of TCG fragments (HCC hypomethylated CpGs) tumor DNA and thus is a good target for enrichment for clinically-relevant DNA.

This behavior for HCC also applies for other cancer types. For examples, DNASE1L3 is downregulated in most cancer types, except for slight increases observed in ESCA and THCA. In contrast, DNASE1 is upregulated in most cancer types, but it is downregulated in ESCA, KIPAN, KIRC, and STES.

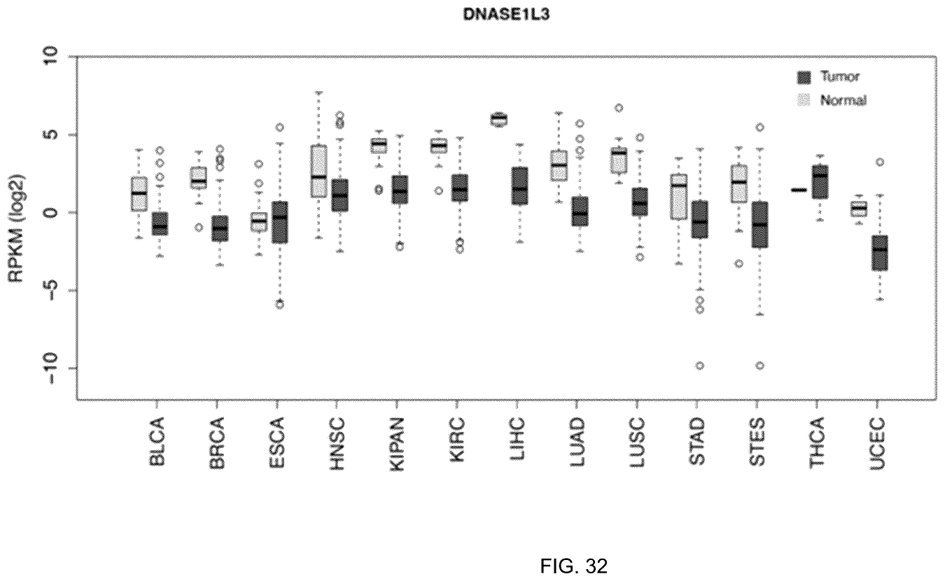

FIG. 32 illustrates the expression level of DNASE1L3 in tumoral tissues and adjacent nontumoral tissues in various of cancers. BLCA: Bladder Urothelial Carcinoma; BRCA: Breast invasive carcinoma; ESCA: Esophageal carcinoma; HNSC: Head and Neck squamous cell carcinoma; KIPAN: Kidney PAN cancer; KIRC: Kidney renal clear cell carcinoma; LIHC: Liver hepatocellular carcinoma; LUAD: Lung adenocarcinoma; LUSC: Lung squamous cell carcinoma; STAD: Stomach adenocarcinoma; STES: Stomach and Esophageal carcinoma; THCA: Thyroid carcinoma; UCEC: Uterine Corpus Endometrial Carcinoma.

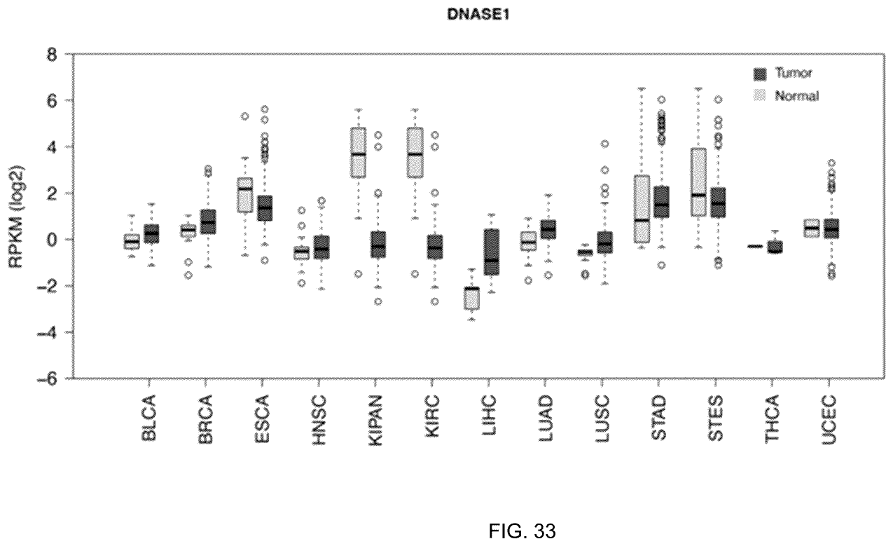

FIG. 33 illustrates the expression level of DNASE1 in tumoral tissues and adjacent nontumoral tissues in various of cancers. BLCA: Bladder Urothelial Carcinoma; BRCA: Breast invasive carcinoma; ESCA: Esophageal carcinoma; HNSC: Head and Neck squamous cell carcinoma; KIPAN: Kidney PAN cancer; KIRC: Kidney renal clear cell carcinoma; LIHC: Liver hepatocellular carcinoma; LUAD: Lung adenocarcinoma; LUSC: Lung squamous cell carcinoma; STAD: Stomach adenocarcinoma; STES: Stomach and Esophageal carcinoma; THCA: Thyroid carcinoma; UCEC: Uterine Corpus Endometrial Carcinoma.

These data further indicate that using fragmentomic features to enrich the targeted cfDNA molecules had the advantage of combining the molecular signals from multiple dimensions, potentially improving the diagnostic performance of cancer detection. And the data indicates applicability for other cancers besides HCC.

### V. Enrichment Using DNA Fragment Size

Clinically-relevant DNA can also be enriched through its methylation-associated cutting positions and fragment size.

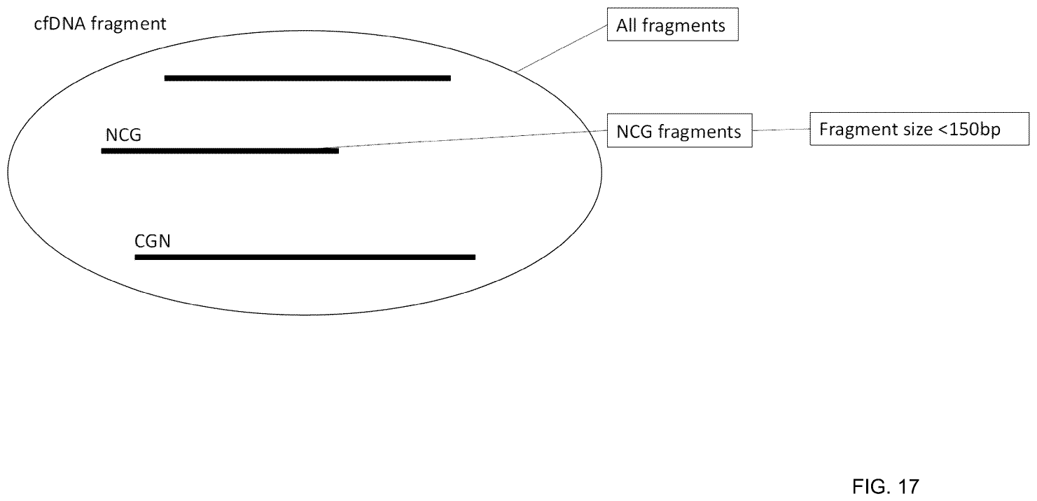

FIG. 17 shows a selection of NCG fragments and a size selection to enrich a sample for clinically-relevant DNA. As shown in FIG. 17, cfDNA fragments carrying NCG or CGN end motifs would be selected. These fragments would be further selected based on the fragment size. For example, cfDNA fragments carrying NCG motif and with a size below a size cutoff (e.g., 150 bp) can be selected for downstream analysis, e.g., fetal DNA fraction or tumor DNA fraction quantification. Other values for the size cutoff can be 70, 80, 90, 100, 110, 120, 130, 140, 160, 170, 180, 190, 200, and 210 bp or any value in between.

### A. Fetal Example

As an example, fetal DNA fractions of all cfDNA fragments, cfDNA with a size <150 bp, and cfDNA carrying NCG end motifs with a size <150 bp in 30 pregnancy samples from different trimesters were analyzed.

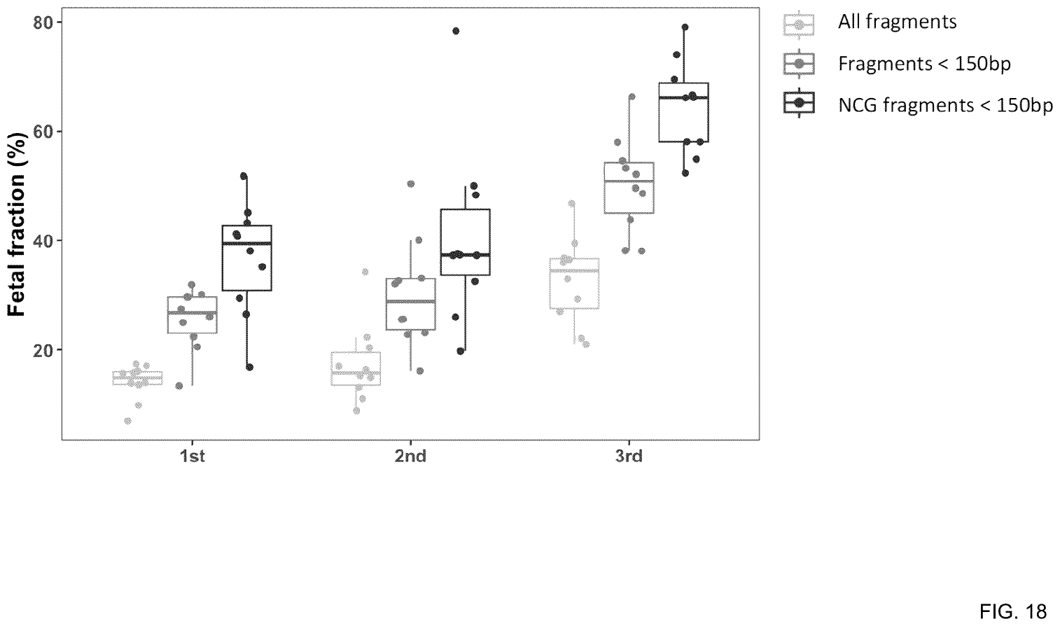

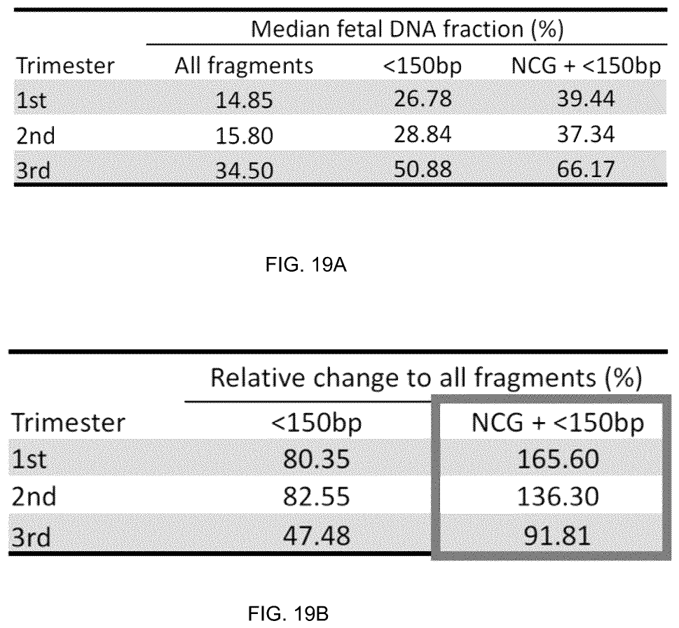

FIG. 18 shows boxplots of the fetal fraction for all DNA fragments, DNA fragments less than 150 bp, and NCG DNA fragments less than 150 bp. FIGS. 19A-19B show tables of the fetal fraction for all DNA fragments, DNA fragments less than 150 bp, and NCG fragments less than 150 bp and relative change to all DNA fragments.

As shown in FIGS. 18-19B, selective analysis of cfDNA fragments carrying NCG end motif and with a size <150 bp showed around 165.60%, 136.30%, and 91.81% increase of fetal DNA fraction in 1st (median fetal fraction: NCG fragments <150 bp: 39.44% vs. all fragments: 14.85%), 2nd (median fetal fraction: NCG fragments <150 bp: 37.34% vs. all fragments: 15.80%), and 3rd (median fetal fraction: NCG fragments <150 bp: 66.17% vs. all fragments: 34.50%) trimesters, respectively. This increase in fetal fraction outperforms using size alone, which indicated around 80.35%, 82.55%, and 47.48% increase of fetal DNA fraction in 1st (median fetal fraction: fragments <150 bp: 26.78% vs. all fragments: 14.85%), 2nd (median fetal fraction: fragments <150 bp: 28.84% vs. all fragments: 15.80%), and 3rd (median fetal fraction: fragments <150 bp: 50.88% vs. all fragments: 34.50%) trimester, respectively (FIGS. 18-19B). These data indicated that the combined use of different fragmentomic features could largely enrich the placenta-derived cfDNA.

### B. Tumor Example

As another example, tumor DNA fractions of all cfDNA fragments, cfDNA with a size <150 bp, and cfDNA carrying NCG end motif with a size <150 bp in one HCC case were analyzed.

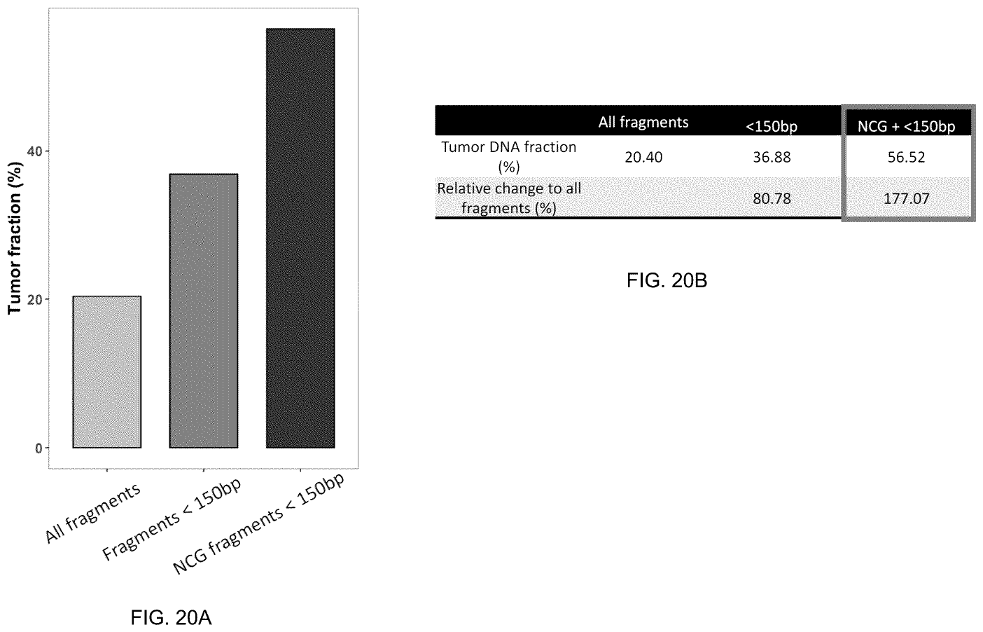

FIG. 20A shows the fetal fraction for all DNA fragments, DNA fragments less than 150 bp, and NCG DNA fragments less than 150 bp. FIG. 20B shows a table of the fetal fraction for all DNA fragments, DNA fragments less than 150 bp, and NCG fragments less than 150 bp and relative change to all DNA fragments.

As shown in FIGS. 20A-20B, selective analysis of cfDNA fragments carrying NCG end motif and with a size <150 bp showed around 177.07% increase in tumor DNA fraction (tumor fraction: NCG fragments <150 bp: 56.52% vs. all fragments: 20.40%). This increase in tumor fraction outperforms that using size alone, which indicated around 80.78% increase in tumor DNA fraction (median fetal fraction: fragments <150 bp: 36.88% vs. all fragments: 20.40%). These data indicated that the combined use of different fragmentomic features could largely enrich the tumor-derived cfDNA.

### VI. Example Assays

Various assays can be used to perform enrichment. Additionally, the enrichment can be performed physically or in silico, e.g., by selecting cfDNA fragments that have specified properties, such as particular end motif (e.g., CGN or NCG, including one or more specific 3-mers), are from a particular part of the genome (e.g., hypomethylated or hypermethylation CpG sites), and/or a particular fragment size. After physical enrichment, the cfDNA can be analyzed can be performed, e.g., using sequencing or probe-based techniques. Below are example assays that perform physical enrichment for particular end motifs and from a particular region. As examples, such assays can use primers and/or probes affixed to surfaces or beads. After enrichment and analysis, a property of the sample or subject may optionally be determined.

### A. Targeted Sequencing

One example technique to enrich clinically-relevant DNA for subsequent analysis is targeted sequencing, e.g., by amplifying DNA fragments having a sequence corresponding at least partly from one or more regions of interest and/or using capture probes that select such DNA fragments. Regions that contain tissue-specific differently methylated CpGs can be used. Certain DNA molecules having particular end motifs corresponding to particular distances from the CpG site to the end of the fragment can be amplified or captured.

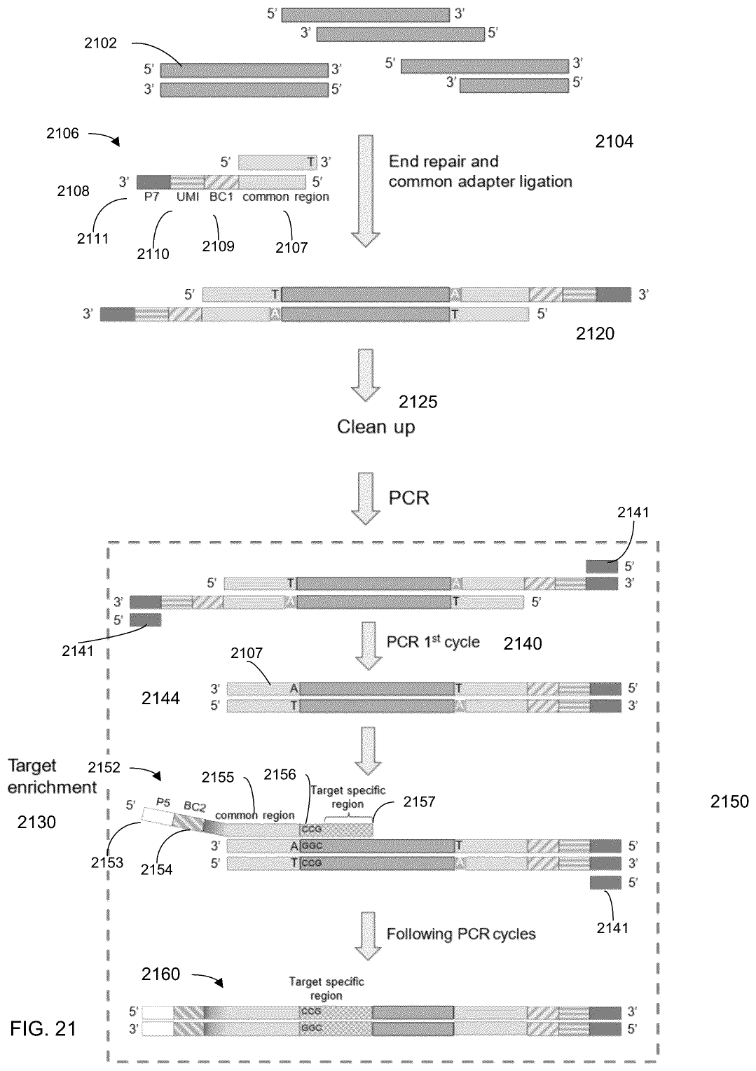

FIG. 21 shows an example workflow for the sequencing library preparation by selectively amplifying cfDNA containing CCG end motifs in which the CG represents a tissue-specific hypomethylated CpG site. A similar protocol can be performed for other end motifs (e.g., all NCG end motifs or any or all of CGN motifs). And hypermethylated CpG sites could be used instead.

As shown, cfDNA molecules 2102 can be subjected to process 2104 of DNA end pair, A-tailing, and common adaptor ligation as optional steps. The end repair can add or subtract nucleotides such that the DNA ends are blunt (i.e., nether strand extends beyond the other strand). The A-tailing may be a result of the use of a particular polymerase. For example, 5′ end phosphorylation and dA-Tailing to the 3′ end may be performed.

For the ligation, an artificial DNA sequence 2106 that includes two strands can be added. A bottom strand 2108 can include one or more of: a common region 2107 (part of original DNA fragment), a barcode 2109 (e.g., corresponding to a particular sample), a unique molecular identifier 2110 (“UMI”) that removes PCR duplicates at later stages, and a common adaptor 2111, which could provide a binding site annealed to the adaptor fixed in the surface of a flow cell and another binding site annealed to sequencing primer, facilitating sequencing in Illumina platform. For example, at the start of Illumina sequencing (when used), bridge amplification is performed; P7 and P5 are two adaptors at the end of the DNA library that are involved in the bridge amplification. Ligated DNA molecules 2120 result.

A cleanup process 2125 can remove reagents remaining from previous steps, including oligonucleotides and polymerases. In some embodiments, this cleanup step can also involve size selection prior to the final library, obtaining DNA fragments with a target size range. At this stage, the size selection is physical selection, e.g., using electrophoresis and/or filtration. Size selection at later stages (after sequencing) can be done via computer analysis, i.e., in silico.

A target enrichment 2130 can be performed using amplification (e.g., PCR) of target DNA. Other amplification techniques could be used instead, e.g., rolling circle or multiple displacement amplification. Alternatively capture probes could be used for enrichment. In a first PCR cycle 2140, a first primer 2141 is used to bind to common adaptor 2111 to extend the reverse strands. Thus, first primer 2141 can be a reverse primer targeting a common end of common adaptor 2111. Extended DNA molecules 2120 result and contain common region 2107, which is a binding site for a second primer 2152.

In the following PCR cycles, first primer 2141 and a second primer 2152 work together to amplify DNA fragments of interest, thereby creating the library through PCR amplification. Second primer 2152 can bind to common region 2107. As shown, second primer 2152 can include one or more of oligonucleotides 2153 used for the sequencing, a barcode 2154 (e.g., corresponding to a particular sample), oligonucleotides 2155 binding to common regions 2107, oligonucleotides 2156 binding to the CCG motif (e.g., CCG end, TCG end, etc.), and oligonucleotides 2157 binding to the specific region of interest.

The final cfDNA product 2160 includes cfDNA fragments with motif end of interest (e.g., CCG) coming from a target specific region. Those libraries would be subsequently sequenced. Such sequenced reads can be used for performing the analysis to determine the presence of pathological signals, such as but not limited to copy number aberrations, mutations, methylation changes, and fragmentomic changes. To estimate how this assay would improve the performance of NIPT, we have performed the computer simulation analysis and determined the number of reads needed to detect trisomy 21 with 100% specificity in one pregnancy case with 5% fetal DNA fraction in the plasma by using whole genome sequencing and the method presented above.

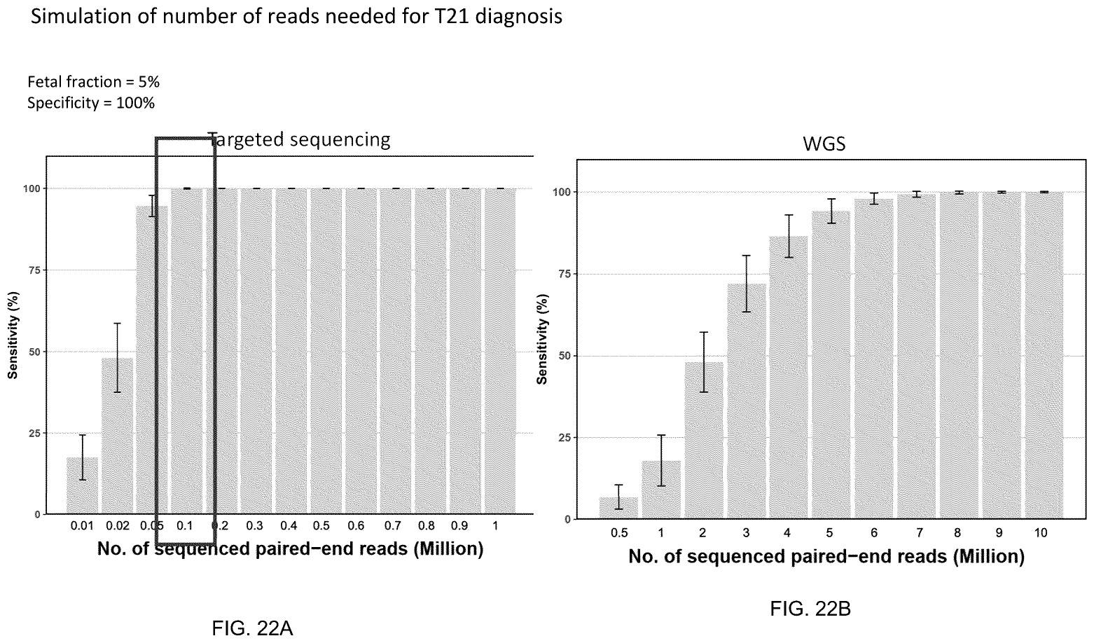

FIGS. 22A-22B shows simulated amount of reads for a Trisomy 21 diagnosis. The fetal fraction is fixed to be 5%, and the expected specificity is 100%. FIG. 22A shows the simulation result for using the targeted sequencing disclosed herein. To achieve the expected 100% specificity, only 0.1 million reads are needed. FIG. 22B shows the simulation result for using the conventional whole genomic sequencing (“WGS”). To achieve have the expected 100% specificity, approximately 10 million reads are needed. Thus, by using the targeted sequencing disclosed herein, the amount of reads can be reduced 100 fold. The targeted sequencing approach can be utilized to enrich clinically-relevant DNA from regions of interest for downstream analysis in a cost-effective manner.

### B. Probe-Based Techniques (e.g., PCR)

The amplification or capture techniques can involve using probes that provide a detection signal. In this manner, the physical enrichment process can also perform detection.

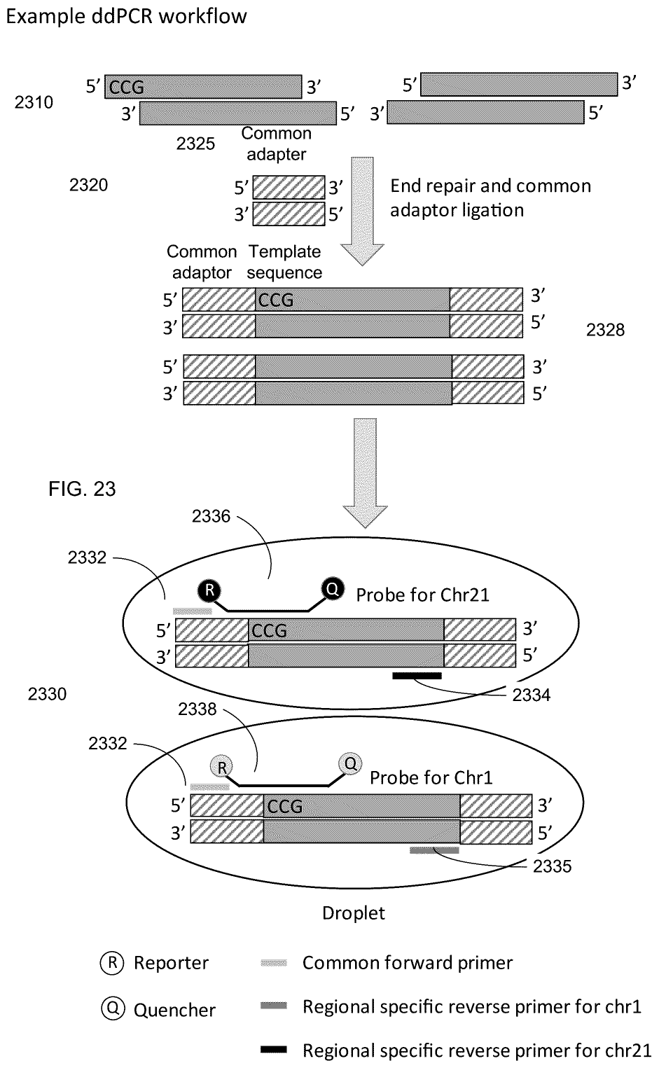

FIG. 23 shows an example workflow using PCR. In various embodiments, digital PCR, such as droplet digital PCR (ddPCR), or qPCR can be used.

In step 2310, cfDNA input is received. The cfDNA input may include a plurality of cfDNA fragments having different fragmentomic features.

In step 2320, end repair and common adaptor ligation adding a common adaptor 2325 are performed on the received cfDNA input, to obtain ligated DNA molecules 2328. Accordingly, the cfDNA molecules can be subjected to the process of DNA end pair, A-tailing (adding an A nucleotide at the end of the fragment), and common adaptor ligation, with the end repair and A-tailing being optional steps. For example, common adaptor 2325 can be ligated by single-strand DNA (ssDNA) ligation without end repair and A-tailing.

The DNA end repair can make the ends blunted so there is no overhang between the two strands. The A-tailing step would facilitate the downstream ligation reaction with the addition of A ends. Common adaptor 2325 can be ligated to the end of the fragments on the basis of a ligase. The adaptor-ligated molecules can be partitioned, e.g., into different reactions, such as droplets, as may be done for digital PCR.

In step 2330, probes 2336 and 2338 targeting DNA template sequences are used. The probes cover the junction of common adaptor and the DNA template sequence. Probes 2336 and 2338 can be used with or without primers, e.g., just in a microarray without any amplification of a particular region. In an embodiment using microarrays without amplification, the probe can be labeled with one or more fluorescent dyes different than the reporter dye shown in FIG. 23, which requires amplification, so the probe can be read by a sensor (e.g., microarray). In this manner, the probes can detect such end motifs anywhere in the genome. In other implementations, a pair of PCR primers can be designed in a way a first primer 2332 (e.g., common forward primer) binds to common adaptor 2325, and second primers 2334 and 2335 (e.g., regional specific reverse primer) could bind to the specific region of interest. The regional specific reverse primers can be from different chromosomes. DNA molecules would be amplified inside a reaction (e.g., droplet) by the pair of PCR primers.

Two different fluorescent probes 2336 and 2338 can be used, e.g., a probe to detect CCG end motif with tissue-specific differently methylated CpG from chromosome 21 (or other chromosome or chromosomal regions being tested for a sequence imbalance) and a probe to detect CCG end motif with tissue-specific differently methylated CpG, e.g., from chromosome 1 or other suitable reference chromosome. The fluorescent probe specific to a particular chromosome (such as chromosome 1 or chromosome 21) can be hydrolyzed and emit fluorescent signals, thus enabling the quantification of copy number aberrations. For digital PCR, the number of reactions positive for a particular chromosome can be counted and used to determine the copy number aberration or other property of the sample. For real-time PCR, the intensity of each signal can be used as a measure of the amount of DNA fragments from different chromosomes. The two intensities can be compared to each other to provide a final measured signal (e.g., a signal ratio) that is compared to a reference value to determine the property of the sample.

Accordingly, different probes and primers are used for chromosome 21 and chromosome 1. For example, the probe used for chromosome 21 covers specific regions from chromosome 21 and the end motif (e.g., NCG) of interest. In this manner, we can obtain an amount of chromosome 21 CCG cfDNA fragments and chromosome 1 CCG cfDNA fragments. In some embodiments, Trisomy 21 diagnosis may be based on counting the chromosome 21 and the chromosome 1 reads, e.g., any parameter define herein, such as a ratio of the two amounts (count or signal intensity). As examples, a direct ratio of x/y is a separation value, as well as x/(x+y).

Further steps of PCR may be performed to determine a property of the biological sample (e.g., whether an aberration exists in a region). When performing real-time PCR, the enriched sample can be placed in one reaction, from which an intensity signal is measured. When performing digital PCR, the enriched sample can be distributed among a set of reactions, where each reaction is analyzed independently.

### VII. Methods

Various methods are provided for enriching a biological sample for clinically-relevant DNA and/or for determining a property of the clinically-relevant DNA of the biological sample. Such a property can be of the subject from which the biological sample was obtained, e.g., a pathology (such as cancer or a copy number aberration) associated with the clinically-relevant DNA.

### A. Using NCG End Motifs

As described above, NCG end motifs can be used to enrich a sample for clinically-relevant DNA. Additional criteria can also be used, such as location in a genome (e.g., at differentially methylated site(s)/loci/region(s)) or fragment size.

### 1. Using Enriched Sample to Determine Property of Sample

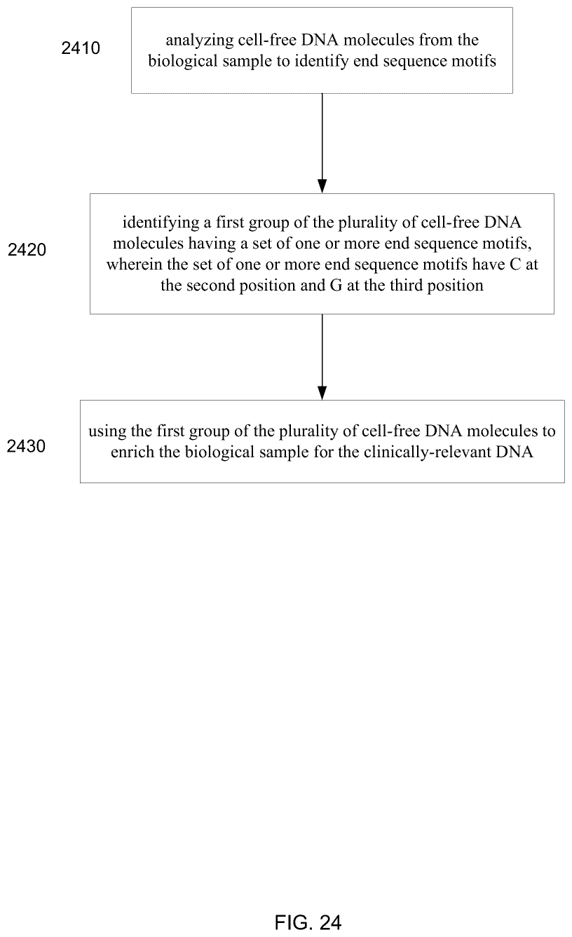

FIG. 24 is a flowchart illustrating a method 2400 of enriching a biological sample of a subject for clinically-relevant DNA using one or more NCG end motifs. The biological sample can include the clinically-relevant DNA and other DNA that are cell-free. In other examples, a biological sample may not include the clinically-relevant DNA (e.g., no tumor DNA may be present). The enriched sample may be a physical sample or an in silico sample (e.g., a set of sequence reads having certain properties). Aspects of method 2400 and any other methods described herein may be performed by a computer system.

At block 2410, a plurality of cell-free DNA molecules from the biological sample of the subject is analyzed. Each cell-free DNA molecule can be analyzed to determining an end sequence motif of at least one end (possibly both ends) of the cell-free DNA molecule. An end of the cell-free DNA molecule has a first position at an outermost position, a second position that is next to the first position, and a third position that is next to the second position.

Various techniques can be used for such analysis in any of the methods described in the present disclosure. For example, the analysis can be performed using sequencing, such as massively parallel sequencing, targeted sequencing, paired-end sequencing, single end sequencing, and single molecule sequencing (e.g., using a nanopore or using real-time single molecule sequencing (e.g., from Pacific Biosciences)), any of which may use a double- or single-stranded DNA sequencing library preparation protocols. The skilled person will appreciate the variety of sequencing techniques that may be used. As part of the sequencing, it is possible that some of the sequence reads may correspond to cellular nucleic acids.

Example techniques can also include probe-based techniques, which can target a particular set of one or more end motifs, as well as potentially target a location (e.g., certain CpG sites as described herein) and a size (e.g., a size range). Such probe-based techniques can include capture or amplification techniques, such as PCR techniques and other mentioned herein. Example PCR techniques can include real-time PCR or digital PCR (e.g., droplet digital PCR). The analysis can include the physical steps of performing such assays and receiving of the measurement data obtained from such assays, or may just include receiving the measurement data.

With any analysis technique, sequence reads of the cell-free DNA molecules can be obtained. The sequence reads can include ending sequences corresponding to ends of the plurality of cell-free DNA fragments. The sequencing may be targeted sequencing as described herein. For example, biological sample can be enriched for DNA fragments from a particular region. The enriching can include using capture probes or primers (e.g., for amplification) that bind to a portion of, or an entire genome, e.g., as defined by a reference genome.

Analyzing a cell-free DNA molecule can include determining a genomic position (location) in a reference genome corresponding to the cell-free DNA molecule (e.g., for at least one end). For example, one or more sequence reads of a DNA molecule (e.g., paired reads at the ends or a read for the entire molecule) can be aligned to the reference genome using any of various alignments techniques as will be appreciated by the skilled person. The alignment can be to some or all of the reference genome. As another example, probe-based techniques can identify a DNA molecule as being from a particular position, e.g., by emitting a particular color for a particular probe that corresponds to a particular genomic position. The position determination can be to some or all of the reference genome, e.g., if only part of the genome is being analyzed. As examples, the amount of the genome analyzed can be greater than 0.01%, 0.1%, 1%, 5%, 10%, 20%, 30%, 40, or 50%, or less than any of such values. Such an analysis may be performed for other methods described herein.

As with other methods described herein, analyzing the plurality of cell-free DNA molecules can includes measuring a size of the cell-free DNA molecule, e.g., as described in FIGS. 17-20. The measurement can be performed in various ways, e.g., using physical separation (such as electrophoresis) and/or sequencing (such as whole molecule sequencing or alignment using paired-end reads). Accordingly, analyzing the cell-free DNA molecules can include determining a size of the cell-free DNA molecule. The sizes can be used to filter the first group of cell-free DNA molecules to obtain cell-free DNA molecules (e.g., as a subgroup) that are smaller than a size cutoff to enrich the biological sample for the clinically-relevant DNA. In various examples, the size cutoff is 500 bp or less. For example, the size cutoff can be 150 bp, where the filtering obtains cell-free DNA molecules less than 150 bp. As another example, the size cutoff can be 300 bp.

Locations (e.g., genomic positions) of the first group of the plurality of cell-free DNA molecules in a reference genome can also be determined, e.g., as described in sections II and IV. A first subgroup of the first group of cell-free DNA molecules can be identified that are located at one or more specified loci to enrich the biological sample for the clinically-relevant DNA (e.g., fetal DNA, tumor DNA, or transplant DNA). The one or more specified loci can be one or more CpG sites that are differentially methylated in the clinically-relevant DNA relative to the other DNA. For example, the one or more CpG sites can be hypomethylated or hypermethylated relative to the other DNA.

When the clinically-relevant DNA is tumor DNA, the one or more CpG sites can include a first set of CpG sites that are hypomethylated relative to the other DNA and a second set of CpG sites that are hypermethylated relative to the other DNA.

The analyzing and the identifying can include physical steps. For example, the analyzing and the identifying can include using a set of one or more oligonucleotides that hybridize to the set of one or more end sequence motifs, e.g., as described in section VI. When a subgroup of the DNA fragments are desired at one or more specified loci, the same set or a different set of one or more oligonucleotides can be used that hybridize to the chromosomal region at the one or more specified loci.

The set of one or more oligonucleotides can be probes, which may be used in one or more PCR reactions, e.g., as described in FIG. 23. Accordingly, when select regions are amplified, the analyzing and the identifying can include using a set of one or more probe oligonucleotides that hybridize to the set of one or more end sequence motifs, where the set of one or more probe oligonucleotides are used in one or more PCR reactions. The one or more PCR reactions can include primers that select regions that include the one or more specified loci. As another example and as described in FIG. 22, the set of one or more oligonucleotides can be part of primers used in one or more PCR reactions. The cell-free DNA products resulting from the one or more PCR reactions can then be sequenced. Such techniques can also be used for other methods described herein

At block 2420, a first group of the plurality of cell-free DNA molecules having a set of one or more end sequence motifs is identified. The set of one or more end sequence motifs can have C at the second position and G at the third position. Examples end sequence motifs include ACG, CCG, GCG, and TCG, collectively referred to as NCG. Longer end motifs (e.g., with K equal to 4 or greater) Any one of more of such end sequence motifs can be used, including just one end sequence motif.

When the clinically-relevant DNA is fetal DNA, the one end sequence motif can have C at the first position (i.e., CCG). When the clinically-relevant DNA is tumor DNA, the one end sequence motif can have T at the first position (i.e., TCG).

At block 2430, the first group of the plurality of cell-free DNA molecules is used to enrich the biological sample for the clinically-relevant DNA. The enriched sample can be created in various ways. For example, when physical steps are performed, the other DNA molecules (i.e., not in the first group) can be washed away. In other implementations, the first group can be amplified.

After enrichment, the first group (or a subgroup, e.g., after filtering) of cell-free DNA molecules of the enriched sample can be used to determine a property of the clinically-relevant DNA of the biological sample. Various properties can be determined, e.g., a copy number aberration of a chromosomal region, mutations, aberrant methylation (and other types of cancer-associated changes), a level of a pathology of the subject (e.g., cancer) where the level of pathology is associated with the clinically-relevant DNA, haplotype inheritance of a fetus, a level of a pathology of a fetus (e.g., an aneuploidy of a chromosome or a chromosomal region or other genetic disorder), or a gestational age of a fetus of a pregnant female from whom the biological sample was obtained.

In some embodiments for determining the property, a first value of the first group of cell-free DNA molecules can be calculated. The first value can define a characteristic of the first group of cell-free DNA molecules and be compared a reference value to determine the property. The reference value can be determined using one or more reference samples having a known property (classification), e.g., (1) subject known to have cancer and known to not have cancer or (2) healthy pregnancies and pregnancies with a sequence imbalance, such as an aneuploidy of a chromosome or a chromosomal region (also referred to as an amplification or a deletion).

In embodiments where a first subgroup located at one or more specified loci is identified, a plurality of specified loci can comprise a chromosomal region, which may include an entire chromosome. A first value defining a characteristic of the first subgroup of cell-free DNA molecules can be calculated and compared to a reference value to determine a classification of whether the chromosomal region exhibits a deletion or an amplification, similar as is described above for a property more generally. The reference value can be determined using samples that have a known classification.

As examples, the characteristic can be a count, a methylation level, or a statistical value of a size distribution. Further details on usage of such characteristics and determinations of properties can be found in U.S. Publication Nos. 2009/0087847, 2009/0029377, 2011/0276277, 2011/0105353, 2013/0040824, and 2018/0216191. For example, the biological sample can be distributed among a set of reactions that are each analyzed independently, e.g., for sequencing or digital PCR. The first value can include a number of reactions that are positive for one or more cell-free DNA molecules having one of the set of one or more end sequence motifs, and potentially hybridizing to one or more specific loci (e.g., part of or all of a chromosomal region that is being tested, such as chromosome 21). In other embodiments, the biological sample can be placed in one reaction, e.g., as in real-time PCR. The first value can be a measure of an intensity signal that is proportional to a number of cell-free DNA molecules having one of the set of one or more end sequence motifs, and potentially hybridizing to one or more specific loci (e.g., part of or all of a chromosomal region that is being tested, such as chromosome 21).

### 2. Determining CNA Using Enriched Sample

As a property, some embodiments can determine whether the clinically-relevant DNA have a copy number aberration (i.e., a deletion or an amplification) in a particular region.

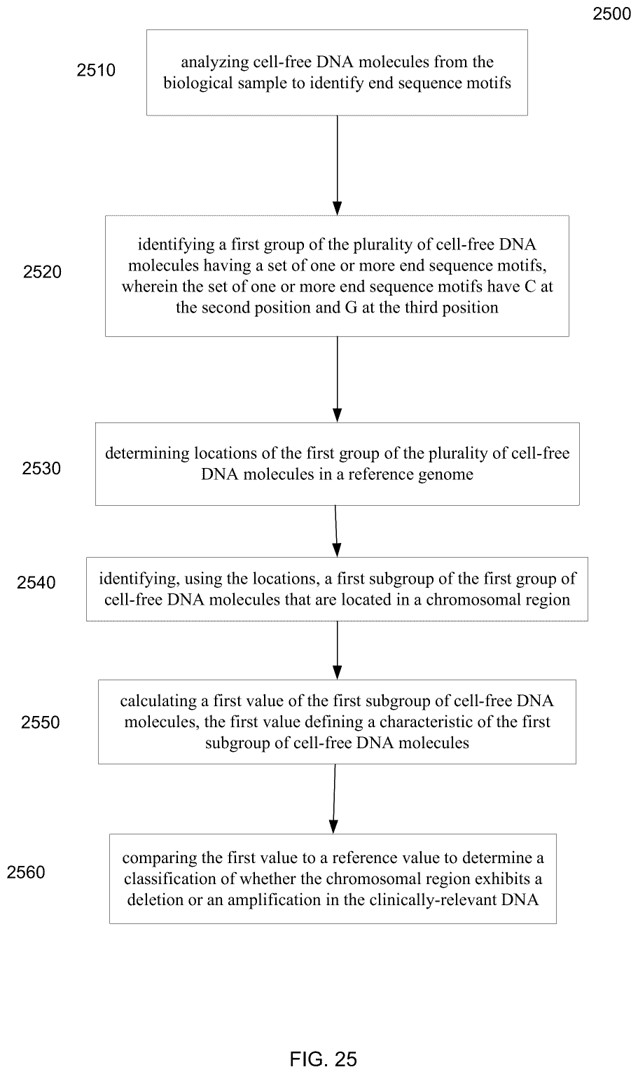

FIG. 25 is a flowchart illustrating a method 2500 of analyzing a biological sample of a subject for genomic deletions or amplifications. The biological sample can include clinically-relevant DNA and other DNA that are cell-free. Aspects of method 2500 can be performed in a similar manner as method 2400.

At block 2510, a plurality of cell-free DNA molecules from the biological sample of the subject are analyzed. Block 2510 may be performed in a similar manner as block 2410. An end sequence motif of at least one end of the cell-free DNA molecule can be determined, where an end of the cell-free DNA molecule has a first position at an outermost position, a second position that is next to the first position, and a third position that is next to the second position.

When physical steps are performed, the analyzing and the identifying can include using a set of one or more oligonucleotides that hybridize to the set of one or more end sequence motifs and that hybridize to the chromosomal region, e.g., as described in section VI. As another example, a first set of one or more oligonucleotides can hybridize to the set of one or more end sequence motifs and a second set of one or more oligonucleotides can hybridize to the chromosomal region. Thus, different primers/probes can be used or the same ones can perform both functions.

At block 2520, a first group of the plurality of cell-free DNA molecules having a set of one or more end sequence motifs is identified. The set of one or more end sequence motifs can have C at the second position and G at the third position, corresponding to NCG. Block 2520 may be performed in a similar manner as block 2420.

At block 2530, locations of the first group of the plurality of cell-free DNA molecules in a reference genome are determined. The locations can be determined in a similar manner as described for method 2400. For example, one or more sequence reads can be aligned to a reference genome. As another example, probe molecule(s) can be used, e.g., as described in section VI.B.

At block 2540, the locations are used to identify a first subgroup of the first group of cell-free DNA molecules that are located in a chromosomal region including one or more specified loci. The loci can be specified in that the chromosomal region is specified, or the loci can be specified to be particular positions or sites within the region. For example, the one or more specified loci can be one or more CpG sites that are differentially methylated in the clinically-relevant DNA relative to the other DNA, e.g., hypomethylated. The chromosomal region can be an entire chromosome, e.g., if an aneuploidy is being detected or a smaller region if a subchromosomal amplification or deletion is being detected. As examples, the identifying can use mapping of sequence reads or use of signals, e.g., fluorescent signals from probes.

At block 2550, a first value of the first subgroup of cell-free DNA molecules is calculated. The first value can define a characteristic of the first subgroup of cell-free DNA molecules, e.g., as described above for method 2400.

At block 2560, the first value is compared to a reference value to determine a classification of whether the chromosomal region exhibits a deletion or an amplification in the clinically-relevant DNA. The reference value can be determined from one or more reference samples for whom the classification is known, e.g., for the particular chromosomal region being analyzed. Block 2560 may be performed in a similar manner as block 2460. For example, one reaction can be used (e.g., for real-time PCR) or multiple reactions can be used (e.g., as in digital PCR or sequencing).

### B. Using CGN End Motifs at Hypermethylated Sites

As described above, e.g., in section III, CGN end motifs at hypermethylated sites can be used to enrich a sample for clinically-relevant DNA. Additional criteria can also be used, such as location in a genome (e.g., at differentially methylated site(s)/loci/region(s)) or fragment size. The technique using CGN end motifs can be performed in a similar manner as the techniques for NCG end motifs, and thus certain description is not repeated.

### 1. Using Enriched Sample to Determine Property of Sample

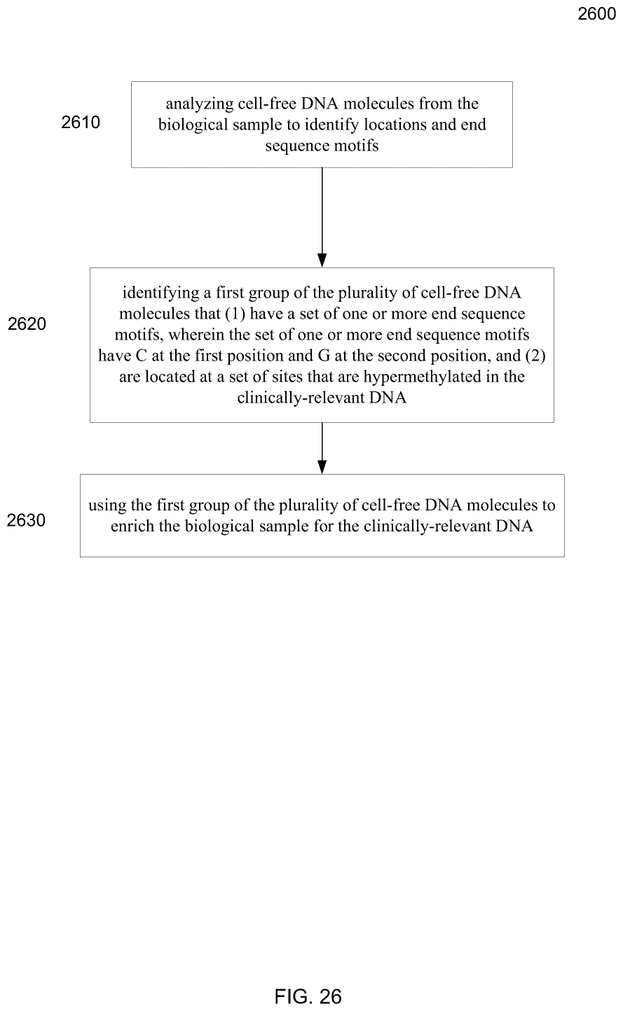

FIG. 26 is a flowchart illustrating a method 2400 of enriching a biological sample of a subject for clinically-relevant DNA using one or more CGN end motifs and tissue-specific hypermethylated sites. Aspects of method 2600 can be performed in a similar manner as methods 2400 and 2500.

At block 2610, a plurality of cell-free DNA molecules from the biological sample of the subject is analyzed. Aspects of block 2610 can be performed in a similar manner as block 2410. Each cell-free DNA molecule can be analyzed to determining an end sequence motif of at least one end (possibly both ends) of the cell-free DNA molecule. An end of the cell-free DNA molecule has a first position at an outermost position, a second position that is next to the first position, and a third position that is next to the second position.

Locations (e.g., genomic positions) of the first group of the plurality of cell-free DNA molecules in a reference genome can also be determined, e.g., as described in sections II and IV.

At block 2620, a first group of the plurality of cell-free DNA molecules is identified. The first group (1) can have a set of one or more end sequence motifs and (2) can be located at a set of sites that are hypermethylated in the clinically-relevant DNA. The set of one or more end sequence motifs can have C at the first position and G at the second position, corresponding to CGN. Aspects of block 2620 can be performed in a similar manner as block 2420.

At block 2630, the first group of the plurality of cell-free DNA molecules is used to enrich the biological sample for the clinically-relevant DNA. Aspects of block 2630 can be performed in a similar manner as block 2430. The enriched sample can be created in various ways. For example, when physical steps are performed, the other DNA molecules (i.e., not in the first group) can be washed away. In other implementations, the first group can be amplified.

Methods using CGN (e.g., method 2600) can be combined with methods using NCG (e.g., methods 2400 and 2500). For example, a second group of the plurality of cell-free DNA molecules can be identified that (1) have another set of one or more end sequence motifs, where the set of one or more end sequence motifs have C at the second position and G at the third position, and (2) are located at another set of sites that are hypomethylated in the clinically-relevant DNA. The second group of the plurality of cell-free DNA molecules can then also be used to enrich a sample and/or to determine a CNA.

### 2. Determining CNA Using Enriched Sample

As a property, some embodiments can determine whether the clinically-relevant DNA have a copy number aberration (i.e., a deletion or an amplification) in a particular region.

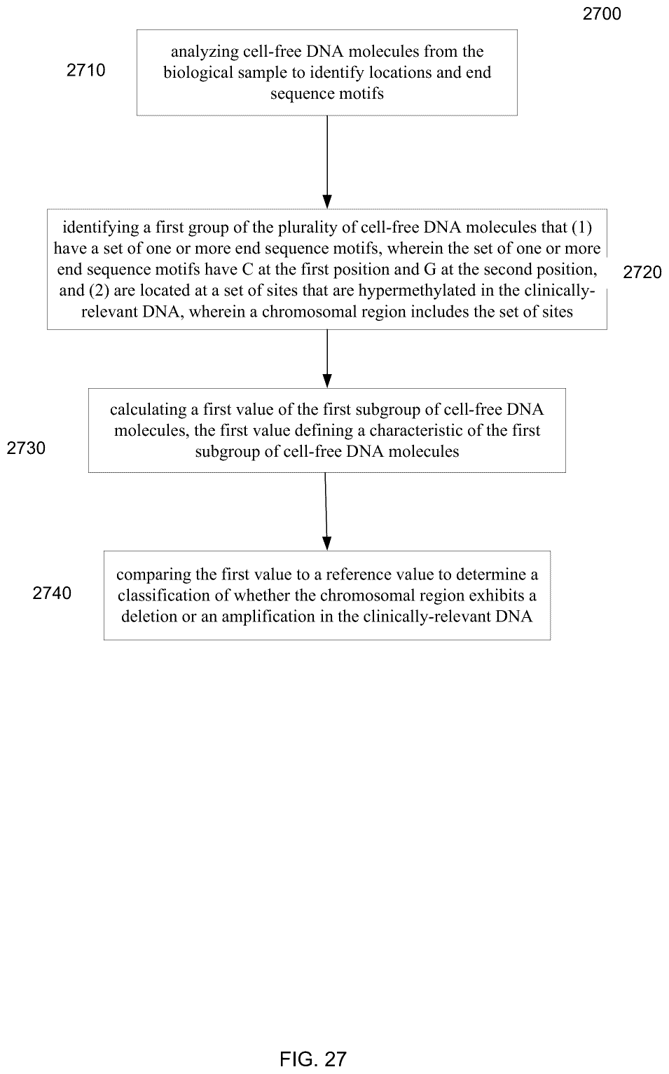

FIG. 27 is a flowchart illustrating a method 2700 of analyzing a biological sample of a subject for genomic deletions or amplifications using one or more CGN end motifs and tissue-specific hypermethylated sites. The biological sample can include clinically-relevant DNA and other DNA that are cell-free. Aspects of method 2700 can be performed in a similar manner as methods 2400, 2500, and 2600.

At block 2710, a plurality of cell-free DNA molecules from the biological sample of the subject are analyzed. Block 2710 may be performed in a similar manner as blocks 2410, 2510, and 2610. An end sequence motif of at least one end of the cell-free DNA molecule can be determined, where an end of the cell-free DNA molecule has a first position at an outermost position, a second position that is next to the first position, and a third position that is next to the second position. A location of in a reference genome can also be determined for the each of the plurality of cell-free DNA molecules.

At block 2720, a first group of the plurality of cell-free DNA molecules is identified. The first group (1) can have a set of one or more end sequence motifs and (2) can be located at a set of sites that are hypermethylated in the clinically-relevant DNA. The set of one or more end sequence motifs can have C at the first position and G at the second position. A chromosomal region includes the set of sites. Block 2720 may be performed in a similar manner as blocks 2420, 2520, and 2620.

At block 2730, a first value of the first group of cell-free DNA molecules is calculated. The first value can define a characteristic of the first group of cell-free DNA molecules, e.g., as described above for method 2400. Block 2730 may be performed in a similar manner as block 2550.

At block 2740, the first value is compared to a reference value to determine a classification of whether the chromosomal region exhibits a deletion or an amplification in the clinically-relevant DNA. The reference value can be determined from one or more reference samples for whom the classification is known, e.g., for the particular chromosomal region being analyzed. Block 2740 may be performed in a similar manner as block 2560. For example, one reaction can be used (e.g., for real-time PCR) or multiple reactions can be used (e.g., as in digital PCR or sequencing). The chromosomal region can be an entire chromosome, e.g., if an aneuploidy is being detected or a smaller region if a subchromosomal amplification or deletion is being detected.

### VIII. Increases in Accuracy for Detecting CNVs

The following data illustrates increasing accuracy for detecting copy number variations (CNVs) using techniques described herein. One example technique to enrich clinically-relevant DNA for subsequent analysis is targeted sequencing. Certain DNA molecules having particular end motifs corresponding to particular distances from the CpG site to the end of the fragment can be amplified.

### A. Fetal Example

We constructed a sequencing library preferentially targeting those fragments carrying CCG motifs (referred to as CCG target sequencing library) for a plasma cfDNA sample obtained from a 3rd trimester pregnant woman, following the example workflow illustrated in FIG. 21. Such CCG target sequencing library was constructed by selectively amplifying cfDNA containing CCG end motifs.

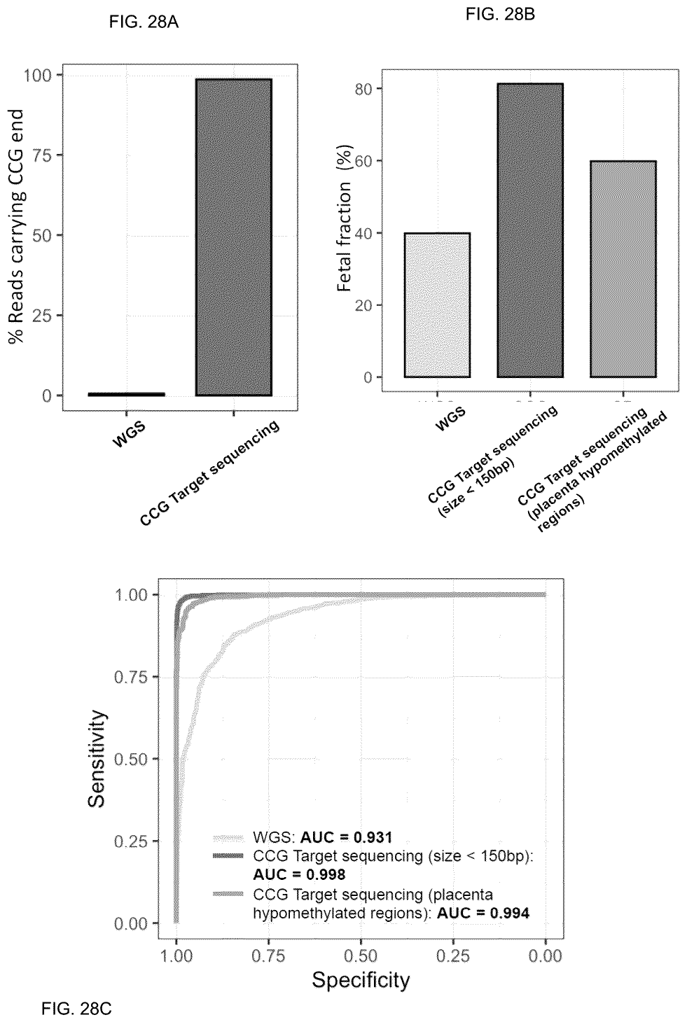

FIGS. 28A-28C show performance of CCG target sequencing in NIPT. FIG. 28A shows a frequency of fragments carrying CCG end motif in whole genome sequencing and CCG target sequencing. FIG. 28B shows a fetal fraction (%) in fragments from whole genome sequencing, from CCG target sequencing with size <150 bp, and from CCG target sequencing with further region selection (i.e., placenta hypomethylated region). FIG. 28C shows simulated ROC curves for detecting trisomy 21 using whole genome sequencing, CCG target sequencing with size <150 bp, and CCG target sequencing with further region selection (i.e., placenta hypomethylated region) with 1 million reads.

As shown in FIG. 28A, selective amplification led to a 196-fold increase in cfDNA fragments carrying CCG end motifs compared to whole genome sequencing. Specifically, the percentage of cfDNA molecules carrying CCG was 98% by using the CCG target sequencing, while it was only 0.5% by whole genome sequencing.

In FIG. 28B, the fetal DNA fraction in fragments with a size <150 bp in CCG target sequencing was 81.25%, while it was 39.89% by whole genome sequencing. Hence, the relative increase in the fetal DNA fraction for CCG target sequencing (size <150 bp) is 103.7%, compared with whole genome sequencing. Based on the relative fetal fraction change compared to all fragments presented in FIG. 13B, FIG. 28B shows that the selection of fragments from placenta-specific hypomethylated CpGs through targeted sequencing can increase the fetal fraction from 39.89% (as observed with whole genome sequencing) to approximately 59.83% (as observed with CCG target sequencing of placenta hypomethylated regions). In some examples, the targeted sequencing can be implemented by, but not limited to, PCR amplification, DNA probe-based hybridization, immunoprecipitation, CRISPR-Cas9 enrichment and other techniques for those cfDNA fragments at least partially originated from one or more regions of interest. To evaluate the potential improvement in the performance of non-invasive prenatal testing (NIPT) using this assay, a computer simulation analysis was conducted as fellow.

The number of sequenced reads of plasma DNA derived from a particular chromosome is assumed to follow the binomial distribution. For a pregnant woman carrying a euploid fetus, the proportion of sequenced reads of plasma DNA originating from chromosome 21 (chr21) is denoted as GR21. Among a total sequenced reads (n), the number of reads derived from chr21 (E) would follow the below distribution:

$$E \sim Binom ( n , GR 21 ) , \qquad ( 1 )$$

where ‘Binom’ represents the binomial distribution.

For a pregnant woman carrying a trisomy fetus, the proportion of sequenced reads of plasma DNA originating from chr21 is denoted as GR′21:

$$G {R}_{21}^{′} = ( 1 + \frac{f}{2} ) \times G {R}_{21} , \qquad ( 2 )$$

where f is the fetal DNA fraction in the maternal plasma DNA of a pregnant woman. Among a total sequenced reads (n), the number of reads derived from chr21 (T) would follow the below distribution:

$$T \sim Binom ( n , {GR}^{′} 21 ) , \qquad ( 3 )$$

which could be rewritten as:

$$T \sim B i n o m ( n , ( 1 + \frac{f}{2} ) \times G {R}_{21} ) . \qquad ( 4 )$$

According to the embodiments in this disclosure, the selective analysis of plasma, f would increase to f′, which is governed by the below formula:

$${f}^{′} = ( 1 + α ) \times f , \qquad ( 5 )$$

where α is the relative increase of the fetal DNA fraction after selective analyses proposed in this disclosure. In this scenario, for a pregnant woman carrying a trisomy fetus, among a total sequenced reads (n), the number of reads derived from chr21 (G) would follow the below distribution:

$$G \sim Binom ( n , ( 1 + \frac{( 1 + α ) \times f}{2} ) \times G {R}_{21} ) . \qquad ( 6 )$$

According to (1), (4) and (6), we simulated sequenced reads originating from chr21 for 1000 plasma DNA samples from pregnant women carrying euploid fetuses and those carrying trisomy 21 fetuses, respectively. We simulated 1000 data points followed by CCG target sequencing with size selection (e.g. <150 bp) from those carrying trisomy 21 fetuses. We simulated 1000 data points followed by CCG target sequencing with selection of molecules within placenta-specific hypomethylation regions from those carrying trisomy 21 fetuses. For each sample, the fetal fraction was assumed to be 5%, and the total sequenced reads (n) were assumed to be 1 million.

The receiver operating characteristic (ROC) analysis was used to determine the discriminating power between pregnant women carrying euploid fetuses and those carrying trisomy 21 fetuses. The binomial distributions were generated with the R function rbinom. ROC curves for the groups with and without selective analyses were plotted with R package pROC (version 1.15.3). The DeLong's test was adopted to compare ROC curves to determine if the improvement of trisomy 21 detection using the method with selective analyses is statistically significant compared with that without selective analyses.

As illustrated in FIG. 28C, CCG target sequencing (with fragment sizes <150 bp) and CCG target sequencing of placenta hypomethylated regions achieved AUCs of approximately 0.998 (P<0.01; DeLong test) and 0.994 (P<0.01; DeLong test), respectively, which represents a significant increase compared to whole genome sequencing (AUC: 0.931).

The size cutoff is not limited to 150 bp. Additional sizes were also tested. In CCG target sequencing, the fetal DNA fraction in cell-free DNA (cfDNA) fragments of various sizes was higher compared to whole genome sequencing. In some examples, the fetal DNA fraction was 81.25% for fragments <150 bp, 43.31% for fragments <300 bp, and 40.95% for fragments <500 bp. In contrast, whole genome sequencing yielded a fetal DNA fraction of 39.89%. Therefore, CCG target sequencing could enable a notable increase in the fetal DNA fraction when analyzing cfDNA fragments with a particular size range, such as but not limited to <150 bp, <300 bp, and <500 bp, etc.”

### B. Tumor Example

Using the same method, we also estimate how this assay would improve the performance of cancer detection. A computer simulation analysis was conducted as follows.

We assumed a set of regions (R) commonly have a copy number gain (3 copies) across all cancer patients. The number of sequenced reads of plasma DNA derived from R is assumed to follow the binomial distribution. For a healthy individual, the proportion of sequenced reads of plasma DNA originating from R is denoted as GR. Among a total sequenced reads (n), the number of reads derived from R (H) would follow the below distribution:

$$H \sim Binom ( n , GR ) , \qquad ( 1 )$$

where ‘Binom’ represents the binomial distribution.

For a cancer patient, the proportion of sequenced reads of plasma DNA originating from R is denoted as GR′:

$${GR}^{′} = ( 1 + \frac{t}{2} ) \times GR , \qquad ( 2 )$$

where t is the tumor DNA fraction in the plasma DNA of a cancer patient. Among a total sequenced reads (n), the number of reads derived from R (T) would follow the below distribution:

$$T \sim Binom ( n , {GR}^{′} ) , \qquad ( 3 )$$

which could be rewritten as:

$$T \sim B i n o m ( n , ( 1 + \frac{t}{2} ) \times GR ) . \qquad ( 4 )$$

According to the embodiments in this disclosure, the selective analysis of plasma, t would increase to t′, which is governed by the below formula:

$${t}^{′} = ( 1 + α ) \times t , \qquad ( 5 )$$

where a is the relative increase of the tumor DNA fraction after selective analyses proposed in this disclosure. In this scenario, for a cancer patient, among a total sequenced reads (n), the number of reads derived from R (G) would follow the below distribution:

$$G \sim Binom ( n , ( 1 + \frac{( 1 + α ) \times t}{2} ) \times GR ) , \qquad ( 6 )$$

According to (1), (4) and (6), we simulated sequenced reads originating from R (account for 1% of the whole genome) 1000 plasma DNA samples from healthy individuals and cancer patients carrying copy number gain (3 copies) in these regions (R), respectively. We simulated 1000 data points followed by TCG target sequencing with size selection (e.g. <150 bp) from cancer patients carrying copy number gain (3 copies). We simulated 1000 data points followed by TCG target sequencing with selection of molecules within cancer-specific hypomethylation regions from cancer patients carrying copy number gain (3 copies). For each sample, the tumor fraction was assumed to be 2%, and the total sequenced reads (n) were assumed to be 1 million. The receiver operating characteristic (ROC) analysis was used to determine the discriminating power between healthy individuals and cancer patients carrying copy number gain. The binomial distributions were generated with the R function rbinom. ROC curves for the groups with and without selective analyses were plotted with R package pROC (version 1.15.3). The DeLong's test was adopted to compare ROC curves to determine if the improvement of cancer detection using the method with selective analyses is statistically significant compared with that without selective analyses.

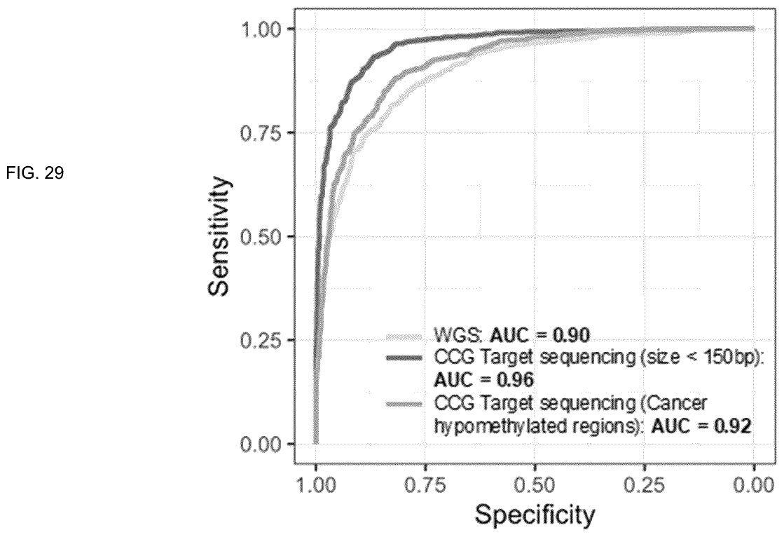

FIG. 29 shows a simulated ROC curves for detecting cancer copy number variation using whole genome sequencing, TCG target sequencing with size <150 bp, and TCG target sequencing with further region selection (i.e., cancer hypomethylated region) with 1 million reads. As illustrated in FIG. 29, TCG target sequencing (with fragment sizes <150 bp) and TCG target sequencing of cancer hypomethylated regions achieved AUCs of approximately 0.96 (P<0.01; DeLong test) and 0.92 (P<0.01; DeLong test), respectively, which represents a significant increase compared to whole genome sequencing (AUC: 0.90).

### IX. Treatments

### A. Further Screening Modalities

Based on any classification, e.g., regarding a pathology or fractional concentration of clinically-relevant DNA, the subject can be referred for additional screening modalities, e.g. using chest X ray, ultrasound, computed tomography, magnetic resonance imaging, or positron emission tomography. Such screening may be performed for cancer.

### B. Treatment Selection

Embodiments of the present disclosure can accurately predict disease relapse, thereby facilitating early intervention and selection of appropriate treatments to improve disease outcome and overall survival rates of subjects. For example, an intensified chemotherapy can be selected for subjects, in the event their corresponding samples are predictive of disease relapse. In another example, a biological sample of a subject who had completed an initial treatment can be sequenced to identify viral DNA that is predictive of disease relapse. In such example, alternative treatment regimen (e.g., a higher dose) and/or a different treatment can be selected for the subject, as the subject's cancer may have been resistant to the initial treatment.

The embodiments may also include treating the subject in response to determining a classification of relapse of the pathology. For example, if the prediction corresponds to a loco-regional failure, surgery can be selected as a possible treatment. In another example, if the prediction corresponds to a distant metastasis, chemotherapy can be additionally selected as a possible treatment. In some embodiments, the treatment includes surgery, radiation therapy, chemotherapy, immunotherapy, targeted therapy, hormone therapy, stem cell therapy, or precision medicine. Based on the determined classification of relapse, a treatment plan can be developed to decrease the risk of harm to the subject and increase overall survival rate. Embodiments may further include treating the subject according to the treatment plan.

### C. Types of Treatments

Embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And, the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment may include resection. For bladder cancer, treatments may include transurethral bladder tumor resection (TURBT). This procedure is used for diagnosis, staging and treatment. During TURBT, a surgeon inserts a cystoscope through the urethra into the bladder. The tumor is then removed using a tool with a small wire loop, a laser, or high-energy electricity. For patients with non-muscle invasive bladder cancer (NMIBC), TURBT may be used for treating or eliminating the cancer. Another treatment may include radical cystectomy and lymph node dissection. Radical cystectomy is the removal of the whole bladder and possibly surrounding tissues and organs. Treatment may also include urinary diversion. Urinary diversion is when a physician creates a new path for urine to pass out of the body when the bladder is removed as part of treatment.

Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

### X. Example Systems

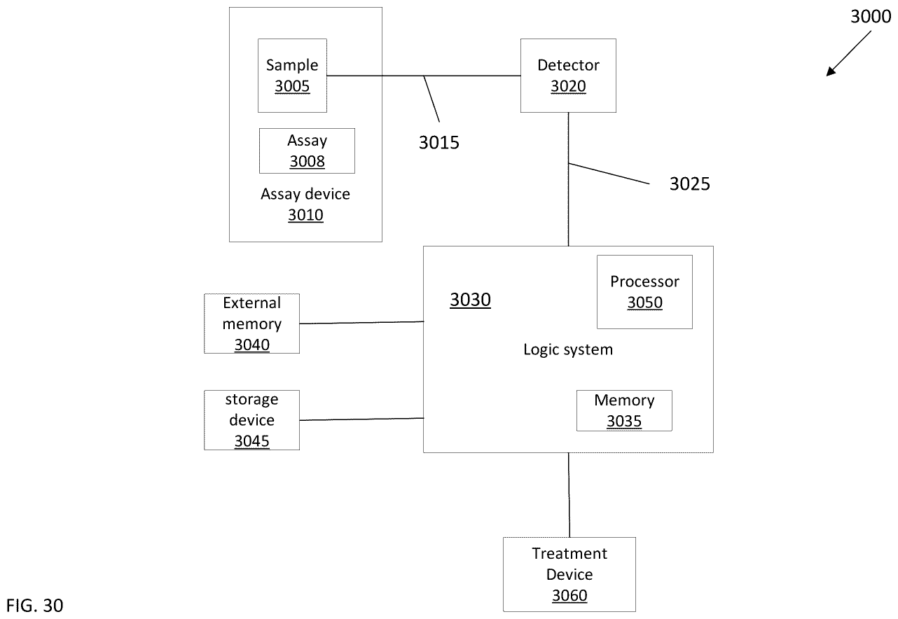

FIG. 30 illustrates a measurement system 3000 according to an embodiment of the present disclosure. The system as shown includes a sample 3005, such as cell-free nucleic acid molecules (e.g., DNA and/or RNA) within an assay device 3010, where an assay 3008 can be performed on sample 3005. For example, sample 3005 can be contacted with reagents of assay 3008 to provide a signal (e.g., an intensity signal) of a physical characteristic 3015 (e.g., sequence information of a cell-free nucleic acid molecule). An example of an assay device can be a flow cell that includes probes and/or primers of an assay or a tube through which a droplet moves (with the droplet including the assay). Physical characteristic 3015 (e.g., a fluorescence intensity, a voltage, or a current), from the sample is detected by detector 3020. Detector 3020 can take a measurement at intervals (e.g., periodic intervals) to obtain data points that make up a data signal. In one embodiment, an analog-to-digital converter converts an analog signal from the detector into digital form at a plurality of times.

Assay device 3010 and detector 3020 can form an assay system, e.g., a sequencing system that performs sequencing according to embodiments described herein. A data signal 3025 is sent from detector 3020 to logic system 3030. As an example, data signal 3025 can be used to determine sequences and/or locations in a reference genome of nucleic acid molecules (e.g., DNA and/or RNA). Data signal 3025 can include various measurements made at a same time, e.g., different colors of fluorescent dyes or different electrical signals for different molecule of sample 3005, and thus data signal 3025 can correspond to multiple signals. Data signal 3025 may be stored in a local memory 3035, an external memory 3040, or a storage device 3045. The assay system can be comprised of multiple assay devices and detectors.

Logic system 3030 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 3030 and the other components may be part of a stand-alone or network connected computer system, or they may be directly attached to or incorporated in a device (e.g., a sequencing device) that includes detector 3020 and/or assay device 3010. Logic system 3030 may also include software that executes in a processor 3050. Logic system 3030 may include a computer readable medium storing instructions for controlling measurement system 3000 to perform any of the methods described herein. For example, logic system 3030 can provide commands to a system that includes assay device 3010 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

Measurement system 3000 may also include a treatment device 3060, which can provide a treatment to the subject. Treatment device 3060 can determine a treatment and/or be used to perform a treatment. Examples of such treatment can include surgery, radiation therapy, chemotherapy, immunotherapy, targeted therapy, hormone therapy, and stem cell transplant. Logic system 3030 may be connected to treatment device 3060, e.g., to provide results of a method described herein. The treatment device may receive inputs from other devices, such as an imaging device and user inputs (e.g., to control the treatment, such as controls over a robotic system).

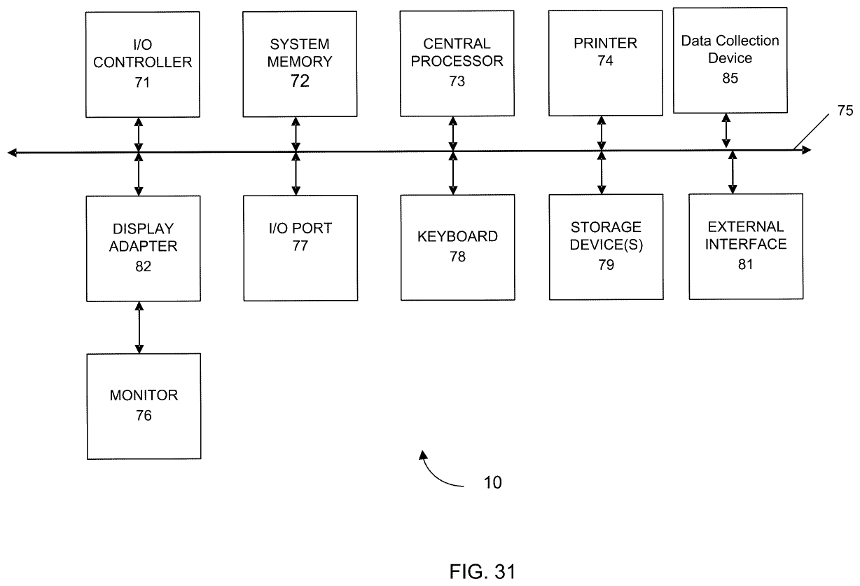

Any of the computer systems mentioned herein may utilize any suitable number of subsystems. Examples of such subsystems are shown in FIG. 31 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 31 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communication messages between devices, including at least 100, 200, 500, 1,000, 10,000, 50,000, 100,000, 500,00, or one million communication messages. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device (e.g., as firmware) or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted to be prior art. Where a conflict exists between the instant application and a reference provided herein, the instant application shall dominate.

What is claimed is:

1. A method of enriching a biological sample of a subject for clinically-relevant DNA, the biological sample including the clinically-relevant DNA and other DNA that are cell-free, the method comprising:
analyzing a plurality of cell-free DNA molecules from the biological sample of the subject, wherein analyzing each cell-free DNA molecule of the plurality of cell-free DNA molecules includes:
determining an end sequence motif of at least one end of the cell-free DNA molecule, wherein an end of the cell-free DNA molecule has a first position at an outermost position, a second position that is next to the first position, and a third position that is next to the second position;
identifying a first group of the plurality of cell-free DNA molecules having a set of one or more end sequence motifs, wherein the set of one or more end sequence motifs have C at the second position and G at the third position; and
using the first group of the plurality of cell-free DNA molecules to enrich the biological sample for the clinically-relevant DNA.

2. The method of claim 1, further comprising:
analyzing the first group of cell-free DNA molecules to determine a property of the clinically-relevant DNA of the biological sample.

3. The method of claim 2, wherein the property of the clinically-relevant DNA of the biological sample is a level of a pathology of the subject.

4. The method of claim 3, wherein the pathology is cancer.

5. The method of claim 2, wherein analyzing the first group of cell-free DNA molecules to determine the property of the clinically-relevant DNA of the biological sample includes:
calculating a first value of the first group of cell-free DNA molecules, the first value defining a characteristic of the first group of cell-free DNA molecules; and
comparing the first value to a reference value to determine the property.

6. The method of claim 1, wherein analyzing each cell-free DNA molecule of the cell-free DNA molecules includes determining a size of the cell-free DNA molecule, the method further comprising:
filtering, using the sizes, the first group of cell-free DNA molecules to obtain cell-free DNA molecules that are smaller than a size cutoff to enrich the biological sample for the clinically-relevant DNA.

7. The method of claim 6, wherein the size cutoff is 500 bp or less.

8. The method of claim 1, further comprising:
determining locations of the first group of the plurality of cell-free DNA molecules in a reference genome; and
identifying, using the locations, a first subgroup of the first group of cell-free DNA molecules that are located at one or more specified loci to enrich the biological sample for the clinically-relevant DNA.

9. The method of claim 1, wherein the analyzing and the identifying include using a set of one or more oligonucleotides that hybridize to the set of one or more end sequence motifs.

10. The method of claim 9, wherein the set of one or more oligonucleotides are probes used in one or more PCR reactions.

11. The method of claim 9, wherein the set of one or more oligonucleotides are part of primers used in one or more PCR reactions, the method further comprising sequencing cell-free DNA products resulting from the one or more PCR reactions.

12. The method of claim 8, wherein the analyzing and the identifying include using a set of one or more probe oligonucleotides that hybridize to the set of one or more end sequence motifs, wherein the set of one or more probe oligonucleotides are used in one or more PCR reactions, and wherein the one or more PCR reactions include primers that select regions that include the one or more specified loci.

13. The method of claim 8, wherein the one or more specified loci are a plurality of specified loci comprising a chromosomal region, wherein the first subgroup of the first group of cell-free DNA molecules are located in the chromosomal region, the method further comprising:
calculating a first value of the first subgroup of cell-free DNA molecules, the first value defining a characteristic of the first subgroup of cell-free DNA molecules; and
comparing the first value to a reference value to determine a classification of whether the chromosomal region exhibits a deletion or an amplification.

14. A method of analyzing a biological sample of a subject for genomic deletions or amplifications, the biological sample including clinically-relevant DNA and other DNA that are cell-free, the method comprising:
analyzing a plurality of cell-free DNA molecules from the biological sample of the subject, wherein analyzing a cell-free DNA molecule includes:
determining an end sequence motif of at least one end of the cell-free DNA molecule, wherein an end of the cell-free DNA molecule has a first position at an outermost position, a second position that is next to the first position, and a third position that is next to the second position;
identifying a first group of the plurality of cell-free DNA molecules having a set of one or more end sequence motifs, wherein the set of one or more end sequence motifs have C at the second position and G at the third position;
determining locations of the first group of the plurality of cell-free DNA molecules in a reference genome;
identifying, using the locations, a first subgroup of the first group of cell-free DNA molecules that are located in a chromosomal region including one or more specified loci;
calculating a first value of the first subgroup of cell-free DNA molecules, the first value defining a characteristic of the first subgroup of cell-free DNA molecules; and
comparing the first value to a reference value to determine a classification of whether the chromosomal region exhibits a deletion or an amplification in the clinically-relevant DNA.

15. The method of claim 8, wherein the one or more specified loci are one or more CpG sites that are differentially methylated in the clinically-relevant DNA relative to the other DNA.

16. The method of claim 15, wherein the clinically-relevant DNA is tumor DNA, and wherein the one or more CpG sites include a first set of CpG sites that are hypomethylated relative to the other DNA and a second set of CpG sites that are hypermethylated relative to the other DNA.

17. The method of claim 15, wherein the one or more CpG sites are hypomethylated relative to the other DNA.

18. The method of claim 1, wherein analyzing includes performing sequencing of the plurality of cell-free DNA molecules to obtain sequence reads.

19. The method of claim 14, wherein the analyzing and the identifying include using a set of one or more oligonucleotides that hybridize to the set of one or more end sequence motifs and that hybridize to the chromosomal region.

20. The method of claim 19, wherein the set of one or more oligonucleotides are probes used in one or more PCR reactions.

21. The method of claim 13, wherein the analyzing and the identifying include using a first set of one or more oligonucleotides that hybridize to the set of one or more end sequence motifs and a second set of one or more oligonucleotides that hybridize to the chromosomal region.

22. The method of claim 21, wherein the biological sample is distributed among a set of reactions, wherein each reaction is analyzed independently, and wherein calculating the first value includes counting a number of reactions that are positive for one or more cell-free DNA molecules having one of the set of one or more end sequence motifs and hybridizing to the chromosomal region.

23. The method of claim 21, wherein the biological sample is placed in one reaction, and wherein calculating the first value includes measuring an intensity signal that is proportional to a number of cell-free DNA molecules having one of the set of one or more end sequence motifs and hybridizing to the chromosomal region.

24. The method of claim 1, wherein the clinically-relevant DNA is fetal DNA.

25. The method of claim 24, wherein the set of one or more end sequence motifs is one end sequence motif, and wherein the one end sequence motif has C at the first position.

26. The method of claim 1, wherein the clinically-relevant DNA is tumor DNA.

27. The method of claim 26, wherein the set of one or more end sequence motifs is one end sequence motif, and wherein the one end sequence motif has T at the first position.

28. The method of claim 5, wherein the characteristic is a count, a methylation level, or a statistical value of a size distribution.

29. A method of enriching a biological sample of a subject for clinically-relevant DNA, the biological sample including the clinically-relevant DNA and other DNA that are cell-free, the method comprising:
analyzing a plurality of cell-free DNA molecules from the biological sample of the subject, wherein analyzing a cell-free DNA molecule includes:
determining a location of the cell-free DNA molecule in a reference genome;
determining an end sequence motif of at least one end of the cell-free DNA molecule, wherein an end of the cell-free DNA molecule has a first position at an outermost position, a second position that is next to the first position, and a third position that is next to the second position;
identifying a first group of the plurality of cell-free DNA molecules that (1) have a set of one or more end sequence motifs, wherein the set of one or more end sequence motifs have C at the first position and G at the second position, and (2) are located at a set of sites that are hypermethylated in the clinically-relevant DNA; and
using the first group of the plurality of cell-free DNA molecules to enrich the biological sample for the clinically-relevant DNA.

30. The method of claim 29, further comprising:
analyzing the first group of cell-free DNA molecules to determine a property of the clinically-relevant DNA of the biological sample.

31. The method of claim 30, wherein the property of the clinically-relevant DNA of the biological sample is a level of a pathology of the subject.

32. The method of claim 31, wherein the pathology is cancer.

33. The method of claim 30, wherein analyzing the first group of cell-free DNA molecules to determine the property of the clinically-relevant DNA of the biological sample includes:
calculating a first value of the first group of cell-free DNA molecules, the first value defining a characteristic of the first group of cell-free DNA molecules; and
comparing the first value to a reference value to determine the property.

34. The method of claim 29, wherein analyzing each cell-free DNA molecule of the cell-free DNA molecules includes determining a size of the cell-free DNA molecule, the method further comprising:
filtering, using the sizes, the first group of cell-free DNA molecules to obtain cell-free DNA molecules that are smaller than a size cutoff to enrich the biological sample for the clinically-relevant DNA.

35. The method of claim 34, wherein the size cutoff is 500 bp or less.

36. The method of claim 29, wherein the analyzing and the identifying include using a set of one or more oligonucleotides that hybridize to the set of one or more end sequence motifs.

37. The method of claim 36, wherein the one or more specified loci are a plurality of specified loci comprising a chromosomal region, wherein the first group of cell-free DNA molecules are located in the chromosomal region, the method further comprising:
calculating a first value of the first group of cell-free DNA molecules, the first value defining a characteristic of the first group of cell-free DNA molecules; and
comparing the first value to a reference value to determine a classification of whether the chromosomal region exhibits a deletion or an amplification.

38. A method of analyzing a biological sample of a subject for genomic deletions or amplifications, the biological sample including clinically-relevant DNA and other DNA that are cell-free, the method comprising:
analyzing a plurality of cell-free DNA molecules from the biological sample of the subject, wherein analyzing a cell-free DNA molecule includes:
determining a location of the cell-free DNA molecule in a reference genome; and
determining an end sequence motif of at least one end of the cell-free DNA molecule, wherein an end of the cell-free DNA molecule has a first position at an outermost position, a second position that is next to the first position, and a third position that is next to the second position;
identifying a first group of the plurality of cell-free DNA molecules that (1) have a set of one or more end sequence motifs, wherein the set of one or more end sequence motifs have C at the first position and G at the second position, and (2) are located at a set of sites that are hypermethylated in the clinically-relevant DNA, wherein a chromosomal region includes the set of sites;
calculating a first value of the first group of cell-free DNA molecules, the first value defining a characteristic of the first group of cell-free DNA molecules; and
comparing the first value to a reference value to determine a classification of whether the chromosomal region exhibits a deletion or an amplification in the clinically-relevant DNA.

39. The method of claim 29, wherein analyzing includes performing sequencing of the plurality of cell-free DNA molecules to obtain sequence reads.

40. The method of claim 38, wherein the analyzing and the identifying include using a set of one or more oligonucleotides that hybridize to the set of one or more end sequence motifs and that hybridize to the chromosomal region.

41. The method of claim 36, wherein the set of one or more oligonucleotides are probes used in one or more PCR reactions.

42. The method of claim 38, wherein the analyzing and the identifying include using a first set of one or more oligonucleotides that hybridize to the set of one or more end sequence motifs and a second set of one or more oligonucleotides that hybridize to the chromosomal region.

43. The method of claim 40, wherein the biological sample is distributed among a set of reactions, wherein each reaction is analyzed independently, and wherein calculating the first value includes counting a number of reactions that are positive for one or more cell-free DNA molecules having one of the set of one or more end sequence motifs and hybridizing to the chromosomal region.

44. The method of claim 40, wherein the biological sample is placed in one reaction, and wherein calculating the first value includes measuring an intensity signal that is proportional to a number of cell-free DNA molecules having one of the set of one or more end sequence motifs and hybridizing to the chromosomal region.

45. The method of claim 29, wherein the clinically-relevant DNA is fetal DNA.

46. The method of claim 45, further comprising:
identifying a second group of the plurality of cell-free DNA molecules that (1) have another set of one or more end sequence motifs, wherein the set of one or more end sequence motifs have C at the second position and G at the third position, and (2) are located at another set of sites that are hypomethylated in the clinically-relevant DNA, wherein the second group of the plurality of cell-free DNA molecules are also used.

47. The method of claim 29, wherein the clinically-relevant DNA is tumor DNA.

48. The method of claim 33, wherein the characteristic is a count, a methylation level, or a statistical value of a size distribution.
